(19) United States
(12) Patent Application Publication
(10) Pub. No.: US 2014/0220554 A1
(43) Pub. Date: Aug. 7, 2014

(54) PROCESSES AND COMPOSITIONS FOR DETECTING HUMAN PAPILLOMA VIRUS TYPES

(71) Applicant: SEQUENOM, INC., San Diego, CA (US)

(72) Inventor: Jay Stoerker, Ann Arbor, MI (US)

(73) Assignee: SEQUENOM, INC., San Diego, CA (US)

(21) Appl. No.: 13/877,902

(22) PCT Filed: Oct. 4, 2011
(86) PCT No.: PCT/US11/54779

§ 371 Date: Dec. 17, 2013

Related U.S. Application Data
(60) Provisional application No. 61/390,116, filed on Oct. 5, 2010.

(51) Int. Cl. C12Q 1/70

(52) U.S. Cl. CPC C12Q 1/708
USPC 435/5

(57) ABSTRACT

Provided herein are compositions and processes that allow for sensitive detection of up to fifteen individual HPV sequences or types in a single, multiplexed test. High risk types that can be detected are HPV 16, 18, 31, 33, 35, 39, 45, 51, 52, 56, 58, 59, 66, 68, and 73. Processes and compositions described herein are based in part on the presence or absence of HPV nucleic acid, including HPV DNA and RNA.

### RELATED PATENT APPLICATIONS

This patent application is a national stage of international patent application number PCT/US2011/054779, filed Oct. 4, 2011, entitled PROCESSES AND COMPOSITIONS FOR DETECTING HUMAN PAPILLOMA VIRUS TYPES, naming Jay Stoerker as an inventor, and designated by Attorney Docket No. SEQ-6028-PC, which claims the benefit of U.S. Provisional Patent Application No. 61/390,116 filed on Oct. 5, 2010, entitled “PROCESSES AND COMPOSITIONS FOR DETECTING HUMAN PAPILLOMA VIRUS TYPES,” naming Jay Stoerker as an inventor, and designated by Attorney Docket No. SEQ-6028-PV. The entire content of the foregoing patent applications are incorporated herein by reference, including all text, tables and drawings.

### FIELD

The technology relates in part to human papilloma virus and methods for detecting the presence, absence or amount of multiple forms of the virus.

### BACKGROUND

Human Papilloma Viruses (HPVs) are small, non-enveloped DNA viruses, approximately 55 nm in diameter, that infect basal cells and replicate in the nucleus of squamous epithelial cells. The genomic organization of each of the papillomaviruses is similar and can be divided into three functional regions. Following infection, the early HPV genes (E6, E7, E1, E2, E4 and E5) are expressed and the viral DNA replicates from the episomal form of the virus. In the upper layer of the epithelium the viral genome is replicated further, and the late genes (L1 and L2) and E4 are expressed. The shed virus can then initiate new infections.

Low-grade intraepithelial lesions support productive viral replication. Progression to high-grade intraepithelial lesions and invasive carcinomas is associated with a persistent high-risk HPV infection and integration of the HPV genome into the host chromosomes, loss or disruption of E2 and subsequent upregulation of E6 and E7 expression. E6 and E7 are the oncogenes of the virus and expression of these genes is required for malignant transformation. Among others, E6 and E7 mediate degradation of the tumor suppressors p53 and RB, respectively, and interfere with cell-cycle regulation. E6 and E7 proteins from low-risk types are less competent in interfering with p53 and pRb functions than E6/E7 proteins from high-risk types. Therefore, low-risk HPV infections are associated with benign proliferations, such as genital warts and low-grade intraepithelial lesions prone to regress.

HPV types 16 and 18 are among the higher risk viral types since their presence is associated with preneoplastic lesions and carcinomas in more than 70% of all cases. There are ten (10) types established in 2007 as carcinogenic by the International Agency for Research on Cancer (IARC), and six (6) other types that are thought to be potentially carcinogenic (Kathrine Lie and Gunnar Kristensen, Expert Rev Mol Diagn. “Human Papillomavirus E6/E7 mRNA Testing as a Predictive Marker for Cervical Carcinoma”, 2008; 8(4):405-415).

In contrast, lower risk types, most commonly HPV types 6 and 11, are typically associated with benign lesions. The oncogenic potential of HPV is principally due to two viral oncoproteins, E6 and E7. Differences in oncogenic potential among HPV types have been attributed to type-specific differences in the E6 and E7 proteins. The E6 protein of oncogenic HPV strains has been shown to interact with the p53 protein and promote its degradation via a ubiquitin-dependent pathway. The E7 oncoprotein can, similarly, complex with the retinoblastoma (Rb) protein and inactivate it. Both p53 and Rb are important tumor suppressor genes whose products regulate the cell cycle, orchestrate DNA repair processes, and are involved with programmed cell death or apoptosis. Disruption of these tumor suppressor proteins by HPV leads to propagation of mutational changes and cell immortalization.

### SUMMARY

Current techniques for detecting HPV are hampered by certain limitations. For example, examining serum DNA for abnormal genomes of cancer cells has been studied as a potential molecular test for cancer. Although some researchers found that the TaqMan quantitative PCR method could detect HPV DNA in serum from some patients with head/neck and cervical cancers, HPV DNA was not detectable by this technique in serum and other biological locations in sufficient amounts for clinical use. Further, certain target capture-based methods, such as hybrid capture assay formats, for example, give rise to the following limitations: (1) cross-reactivity non-specifically with HPV types other than the known high risk types, giving rise to false positives; (2) requirement of at least several thousand HPV molecules to read as positive, preventing screening of serum and/or blood where a smaller number of molecules are present; and (3) not revealing the HPV type found in the cervix, giving rise to false positive results if the types of HPV responsible for a signal are non-high risk types of HPV.

The present technology overcomes such limitations. Provided herein are systems, methods and compositions for detection, identification, and gene expression identification of HPV in biological samples down to the 50 attomolar level. Examples of biological samples include, without limitation, mammalian bodily fluids and cervix scrapings. Results of diagnostic methods provided herein can be utilized for detection, treatment and/or management of cancer and dysplasia. In some embodiments, provided are compositions and processes that allow for sensitive detection of up to fifteen individual HPV sequences or types in a single, multiplexed assay. The presence or absence of HPV nucleic acid, including DNA and RNA, can be assessed using methods and compositions described herein.

Featured in some embodiments are processes and compositions for detection of HPV RNA. Cross-sectional studies have shown that HPV mRNA testing correlates well with the severity of the lesion and appears to be more appropriate for risk evaluation than HPV DNA testing (e.g., Kathrine Lie and Gunnar Kristensen, Expert Rev Mol Diagn. 2008; 8(4):405-415). Assays described herein are not affected by the presence or absence of RNA transcript variants (e.g., assays were designed such that the HPV primers provided do not intersect known HPV E6/E7 RNA transcript splice sites). Thus, provided are methods and compositions that permit screening of bodily fluids and tissues for HPV nucleic acids as a marker of residual tumor or dysplasia in cases associated with HPV.

Also provided in some embodiments are methods for detecting HPV DNA in a biological sample, comprising (a) conducting an amplification reaction of at least a portion of the DNA from the biological sample in the presence of at least one competitor sequence, where the competitor sequence (i) comprises a polynucleotide substantially homologous to a polynucleotide in a DNA sequence of a known HPV type, (ii) includes one or more nucleotide substitutions not present in said HPV DNA sequence, and (iii) is selected from the group consisting of the sequences provided in Table 1D; (b) conducting a primer extension reaction in the presence of at least one extension primer for the known HPV type and at least two different dideoxynucleotides, where at least one extension primer sequence is selected from the group consisting of the sequences provided in Table 1B; and (c) detecting the presence, absence or amount of HPV DNA in a biological sample, where the presence, absence or amount of a known HPV extension products from step (b) is correlated with detecting the presence or absence of HPV DNA. In some embodiments, methods further comprise using amplification primer pairs provided in Table 1A, where the first amplification comprises at least one matched set of forward and reverse primer sequences for the known HPV type, where each primer of the primer pair (i) comprises one of the full length nucleotide sequences hereafter, (ii) comprises one of the non-underlined nucleotide sequences hereafter, or (iii) comprises one of the non-underlined nucleotide sequences hereafter and a tag nucleotide sequence. In certain embodiments, nucleotide substitutions or modifications may be introduced to the sequences described herein. For example, deoxyinosine or other nucleotide substitutes may be introduced. Also, mass modifiers may be used to improve multiplexing.

Also provided in some embodiments are methods for detecting the presence, absence or amount of different high-risk HPV types, for example, one or more selected from the group consisting of HPV16, HPV18, HPV31, HPV33, HPV35, HPV39, HPV45, HPV51, HPV52, HPV56, HPV58, HPV59, HPV66, HPV68 and HPV73. In some embodiments, methods include detecting high-risk HPV DNA in a biological sample containing nucleic acids, and comprise: (a) contacting the nucleic acid of a biological sample with multiple PCR primer pairs provided in Table 1A (e.g., all of the PCR primer pairs in Table 1A); (b) contacting amplification products of step (a) with extend primers comprising a nucleotide sequence selected from Table 1B; and (c) correlating the presence, absence or amount of HPV-specific extension products from step (b) with the presence, absence or amount of high-risk HPV types. In certain embodiments, the HPV types are one or more of the following types selected from the group consisting of HPV16, HPV18, HPV31, HPV33, HPV35, HPV39, HPV45, HPV51, HPV52, HPV56, HPV58, HPV59, HPV66, HPV68 and HPV73. In some embodiments, nucleic acid of the biological sample is amplified in the presence of one or more of the competitor sequences provided in Table 1D.

Certain embodiments are described further in the following description, examples, claims and drawings.

### DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 is a generalized flow diagram of steps in accordance with certain embodiments.

FIG. 2 represents mass spectroscopy results of a screen for fifteen (15) different HPV types with three peaks per type: un-extended primer (UEP.HPV_), wild type (WT) and competitor (Comp), in a single, multiplexed reaction.

FIGS. 3A-3B represents exemplary mass spectroscopy results of HPV detection from a cervical sample for HPV 16. Data for a HPV positive sample (3A) and HPV negative sample (3B) are shown.

FIGS. 4A-4B represents mass spectroscopy results of HPV 16. Competitor of HPV 16 in a normal sample (4A), and Competitor and HPV16 in positive sample (4B).

FIGS. 5A-5P show PCR primer sequences that interrogate E6 gene splice variants. These primer designs allow for amplification of the most widely recognized high risk area for the virus, while avoiding sequence variations that would limit predictive value of the results. Also, the designs are applicable to DNA or RNA sources and include the sequences most type specific. Forward PCR primer is underlined, extension primer is boxed and reverse PCR (polymerase chain reaction) primer is in bold. Nucleotide(s) in italics and bolded represent target single base extension (SBE) nucleotide(s). Double underlined sequences are spliced out of the high risk transcript. The forward PCR primer sequences are underlined and, and reverse PCR primer sequences in bold. The extension primer specified sequences are boxed. The target SBE nucleotide is bold and double underlined. “I” nucleotides represent inosines.

FIGS. 6A-6B represents competitor sequences with a sequence identical to or substantially matching a portion of the nucleic acid sequence of an HPV of interest except for one or more mismatch(es) which are in italics and bolded—used to distinguish between the competitor sequence and template sequence. Forward and Reverse PCR primer specified sequences are underlined. The extension primer specified sequences are boxed. The forward and reverse PCR primer sequences are underlined. The extension primer specified sequences are boxed. The one or more nucleotide mismatch(es) are in italics and bolded, which are used, among other things, to distinguish between the competitor sequence and template sequence.

FIG. 7 represents 5′ DNA modifiers used to add molecular weight to the primers.

### DETAILED DESCRIPTION

Provided in certain embodiments are primer extension-based multiplex HPV nucleic acid amplification assays that can amplify and detect up to fifteen (15) high risk and potential high risk HPV types simultaneously. Such assays are insensitive to HPV 16 variants, and can amplify cDNA made from spliced E6 mRNA of the 15 high risk HPV types at the same time.

Thus, provided in some embodiments are systems, methods, and compositions that can simultaneously analyze and detect the presence, absence or amount of one or more types of high risk HPV. In some embodiments, the analysis and determination can be performed on 100 or fewer HPV copy number, which is more sensitive than tests currently approved by the U.S. Food and Drug Administration (“FDA”) for HPV detection, which require 10,000 copies. Some embodiments further extend the sensitivity by searching for a given individual HPV sequence that enables detection down to 1 attomolar (individual molecules in the 5 microliter PCR volumes used in some embodiments). This increased sensitivity enables the detection of high risk HPV in the blood and serum, among other biological samples.

Provided also are systems, methods, and compositions useful for elaborating details of the type(s) of HPV associated with a given tumor. Such embodiments are sensitive, specific and qualitative, which are not characteristics of certain currently used methods that examine a combination of numerous probes.

In some embodiments, an HPV type or types at the single copy level may be screened sensitively and specifically for detection in biological samples, including without limitation, in mammalian body fluids. Such a sensitive and specific screen makes it well-suited for determination of dysplasia or cancer.

In some embodiments, provided are systems, methods, and compositions that can determine the type and amount of high risk HPV present in a biological sample in a single test. In some embodiments, provided are probes constructed using a mass spectroscopic assay system for one or more high or intermediate risk HPV types. Such high or intermediate risk HPV types may be selected according to identification using the Hologic ThinPrep test, a current FDA-approved test for analysis of HPV in cervical scrapings. Some embodiments are directed to screens that add to the thirteen (13) or less HPV types included in other commercially available tests. Presence, absence or amount of HPV may be determined for 50 attomolar (ca. 300 molecules) of HPV type nucleic acid, an order of magnitude more sensitive than current methods that require several thousand HPV molecules for a positive detection call.

Further, provided in some embodiments are methods that enable the determine whether one or more types of HPV are present in a tumor or dysplasia, or by extension, in materials derived directly from tumors (e.g., cervical ThinPreps). Some embodiments include systems, methods, and compositions for quantitative and/or qualitative analysis. Coupling quantitative determination with ascertainment of HPV type may have significant clinical utility, whereby clinical severity may be reflected by HPV copy number in different anatomic locations.

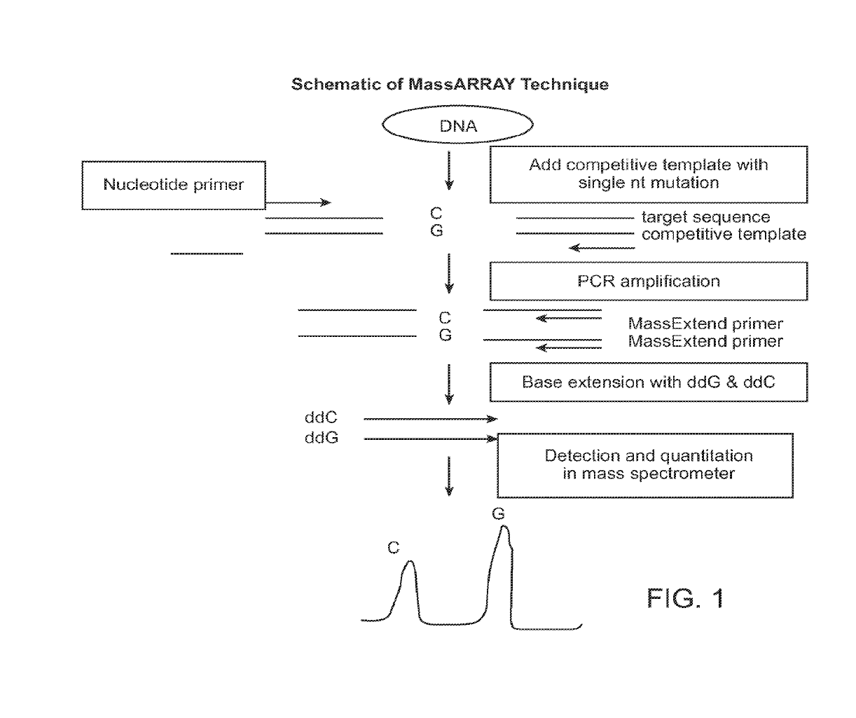

In accordance with some embodiments, the presence of one or more types of high risk HPV in tumor or cellular extracts is detected by a sensitive and specific mass spectroscopic assay (FIG. 1). The mass spectroscopic assay involves, in certain embodiments, amplification by PCR of a short nucleotide fragment found in HPV (when the fragment is present in a sample); digestion of primers and nucleotides; and extension of a “nested” mass spectroscopic assay extend primer that hybridizes to the PCR product (the latter formed when a particular HPV type is present in the sample) with appropriate dideoxynucleotides. In some embodiments, the assay conditions result in incorporation of a single dideoxynucleotide to the mass spectroscopic assay extension sequence only if the given HPV template is present from the first PCR reaction.

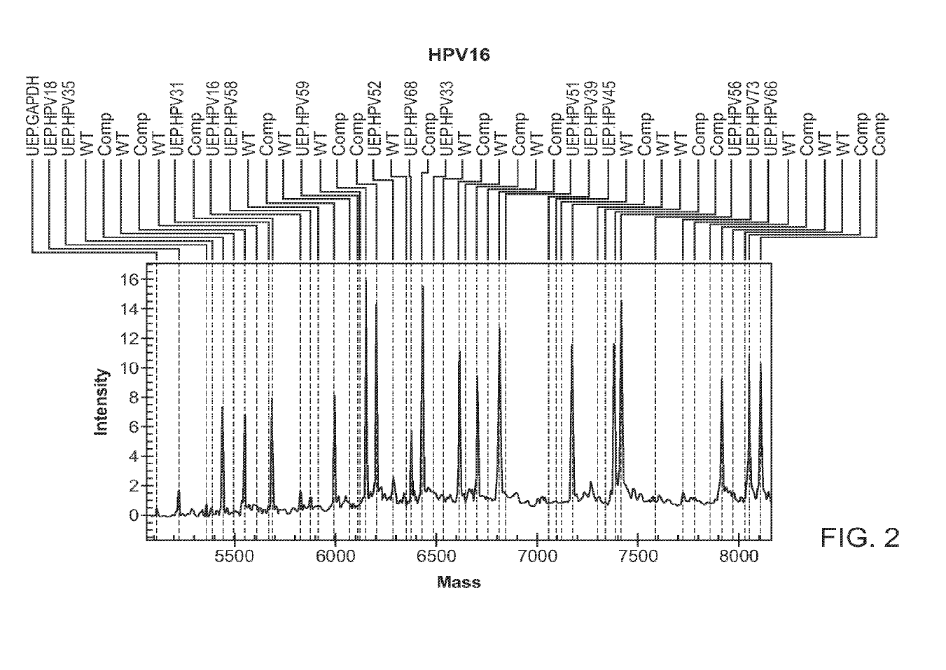

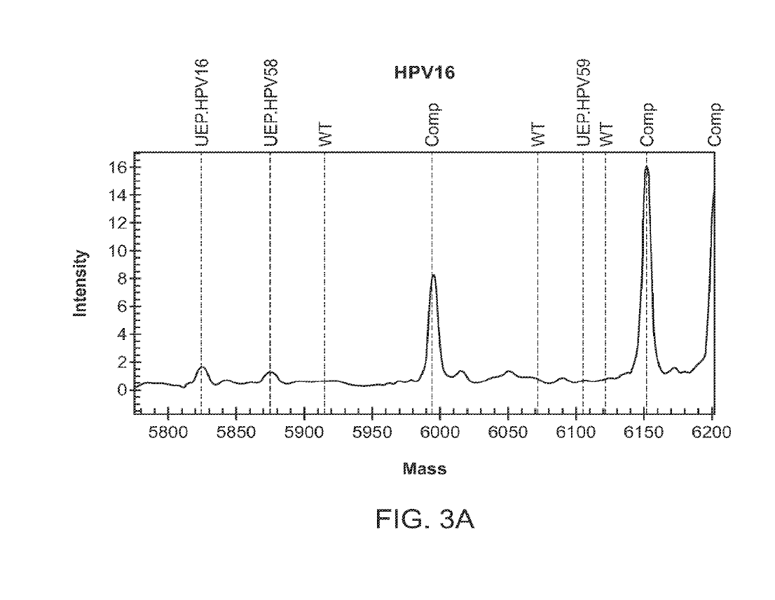

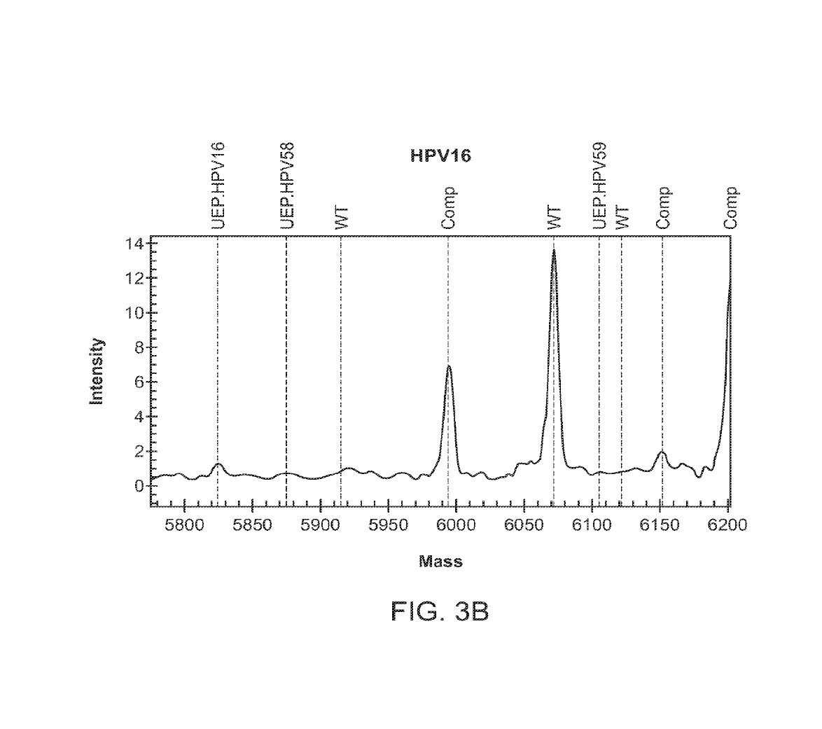

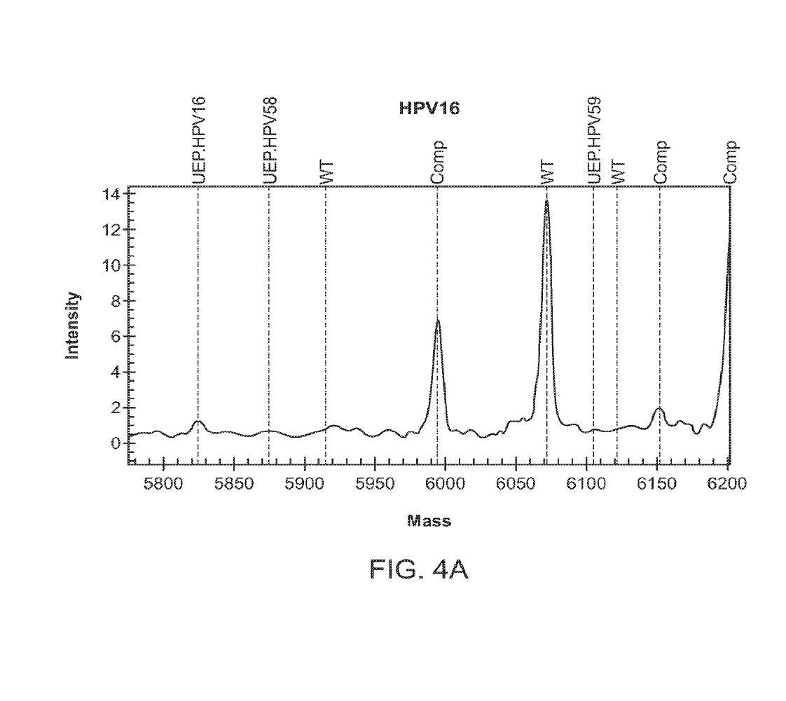

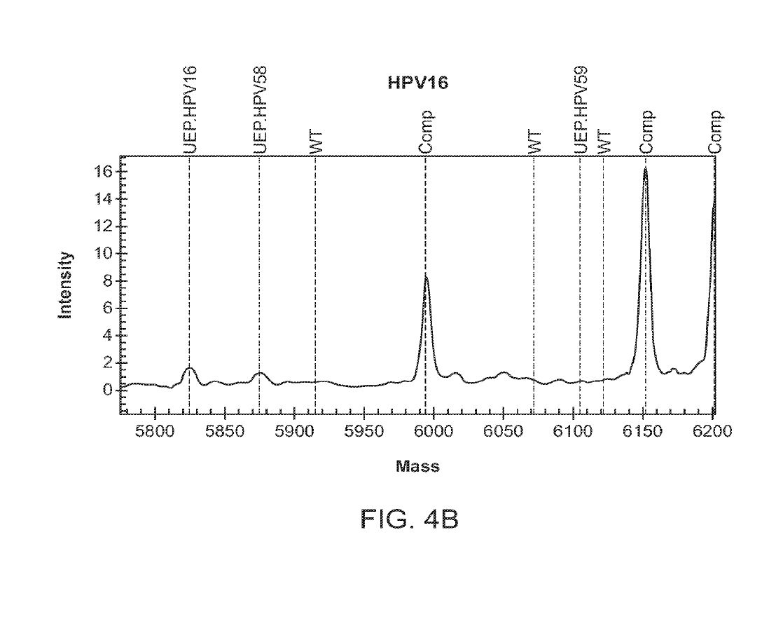

In some embodiments, a screen is set up in a manner where each sample is tested independently for one or more high-risk HPV types. Such screens often are conducted using distinguishable probe(s) that yield a characteristic signal if a sample is positive for a given type of HPV. In certain embodiments, the presence, absence and/or amount of 15 high-risk HPV types are detected (e.g., HPV 16, 18, 31, 33, 35, 39, 45, 51, 52, 56, 58, 59, 66, 68, and 73 (FIG. 2)). In some embodiments, an assay enables screening for a total of up to 15 HPV types that are high risk or potentially high risk. In some embodiments, a screen for one or more of HPV types 6, 11, 16, 18, 31, 33, 35, 39, 45, 51, 52, 56, 58, 59, 66, 68, and/or 73, individually, in any combination with each other, or in combination with other HPV types and/or analytes of interest, is provided.

Some embodiments also include a probe for a single copy fragment of total human genomic DNA (for example, and without limitation, a probe for a single copy fragment of the GAPDH). In addition to highly sensitive screening at least down to the 50 attomolar (aM=10.sup.-18 M) level, assays in certain embodiments permit the determination of the type of HPV associated with a given tumor or dysplasia.

In some embodiments, provided is a two (2) stage screening method that is sensitive and specific enough to detect down to the single molecule level. The first stage involves screening tumorous or dysplastic cells with a battery of primers specific to all 15 high-risk HPV types. Once the type of HPV is known, that type can be used to screen relevant body fluids with greater sensitivity than if all 15 sequences were used simultaneously. As a result, screening of bodily fluids is of increased sensitivity and specificity to have improved clinical utility. Thus, provided are systems, methods and compositions that can detect a HPV type or types in a suitable body fluid (e.g., urine, cerebrospinal fluid, sweat, sputum, tears, buccal swab, etc.). In certain embodiments, the presence, absence and/or amount of HPV DNA and/or RNA is detected.

### Template Nucleic Acid

Template nucleic acid utilized in methods and kits described herein often is obtained and isolated from a subject. A subject can be any living or non-living source, including but not limited to a human, an animal, a plant, a bacterium, a fungus, a protist. Any human or animal can be selected, including but not limited, non-human, mammal, reptile, cattle, cat, dog, goat, swine, pig, monkey, ape, gorilla, bull, cow, bear, horse, sheep, poultry, mouse, rat, fish, dolphin, whale, and shark, or any animal or organism that may have HPV.

Template nucleic acid may be isolated from any type of fluid or tissue from a subject, including, without limitation, umbilical cord blood, chorionic villi, amniotic fluid, cerbrospinal fluid, spinal fluid, lavage fluid (e.g., bronchoalveolar, gastric, peritoneal, ductal, ear, athroscopic), biopsy sample, celocentesis sample, washings of female reproductive tract, urine, feces, sputum, saliva, nasal mucous, prostate fluid, lavage, semen, lymphatic fluid, bile, tears, sweat, breast milk, breast fluid, embryonic cells and fetal cells. In some embodiments, a biological sample may be blood, and sometimes plasma. Fluid or tissue samples often are collected in accordance with standard protocols hospitals or clinics generally follow. A fluid or tissue sample from which template nucleic acid is extracted may be acellular. In some embodiments, a fluid or tissue sample may contain cellular elements or cellular remnants. In some embodiments fetal cells or cancer cells may comprise the sample.

Template nucleic acid can be extracellular nucleic acid in certain embodiments. The term “extracellular template nucleic acid” as used herein refers to nucleic acid isolated from a source having substantially no cells (e.g., no detectable cells; may contain cellular elements or cellular remnants). Examples of acellular sources for extracellular nucleic acid are blood plasma, blood serum and urine. Without being limited by theory, extracellular nucleic acid may be a product of cell apoptosis and cell breakdown, which provides basis for extracellular nucleic acid often having a series of lengths across a large spectrum (e.g., a “ladder”).

The terms “nucleic acid” and “nucleic acid molecule” may be used interchangeably throughout the disclosure. The terms refer to nucleic acids of any composition from, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a non-host organism (e.g., human papillomavirus). The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil. A template nucleic acid may be prepared using a nucleic acid obtained from a subject as a template.

Template nucleic acid may be derived from one or more sources (e.g., cells, soil, etc.) by methods known to the person of ordinary skill in the art. Cell lysis procedures and reagents are commonly known in the art and may generally be performed by chemical, physical, or electrolytic lysis methods. For example, chemical methods generally employ lysing agents to disrupt the cells and extract the nucleic acids from the cells, followed by treatment with chaotropic salts. Physical methods such as freeze/thaw followed by grinding, the use of cell presses and the like are also useful. High salt lysis procedures are also commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, solution 1 can contain 15 mM Tris, pH 8.0; 10 mM EDTA and 100 ug/ml Rnase A; solution 2 can contain 0.2N NaOH and 1% SDS; and solution 3 can contain 3M KOAc, pH 5.5. These procedures can be found in Current Protocols in Molecular Biology, John Wiley & Sons, N.Y., 6.3.1-6.3.6 (1989), incorporated herein in its entirety. Template nucleic acid also may be isolated at a different time point as compared to another template nucleic acid, where each of the samples are from the same or a different source. A template nucleic acid may be from a nucleic acid library, such as a cDNA or RNA library, for example. A template nucleic acid may be a result of nucleic acid purification or isolation and/or amplification of nucleic acid molecules from the sample. Template nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

Template nucleic acid may be provided for conducting methods described herein without processing of the sample(s) containing the nucleic acid in certain embodiments. In some embodiments, template nucleic acid is provided for conducting methods described herein after processing of the sample(s) containing the nucleic acid. For example, a template nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid generally is provided with fewer non-nucleic acid components (e.g., protein, lipid) than the amount of components present in a source sample. A composition comprising isolated template nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to template nucleic acid provided that contains fewer nucleic acid species than in the sample source from which the template nucleic acid is derived. A composition comprising template nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

### Primers

Primers useful for detection, quantification, amplification, sequencing and analysis of HPV nucleotide sequence are provided. In some embodiments a plurality of primer sets, each set comprising pair(s) of primers, may be used. The term “primer” as used herein refers to a nucleic acid that comprises a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence or copy number of a sequence), or feature thereof, for example. A primer may be naturally occurring or synthetic. The term “specific” or “specificity”, as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “primer”, “oligo”, or “oligonucleotide” may be used interchangeably throughout the document, when referring to primers.

A primer nucleic acid can be designed and synthesized using suitable processes, and may be of any length suitable for hybridizing to a nucleotide sequence of interest (e.g., where the nucleic acid is in liquid phase or bound to a solid support) and performing analysis processes described herein. Primers may be designed based upon a target nucleotide sequence. A primer in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer may be composed of naturally occurring and/or non-naturally occurring nucleotides (e.g., labeled nucleotides), or a mixture thereof. Primers suitable for use with embodiments described herein, may be synthesized and labeled using known techniques. Oligonucleotides (e.g., primers) may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides can be effected by native acrylamide gel electrophoresis or by anion-exchange high-performance liquid chromatography (HPLC), for example, as described in Pearson and Regnier, J. Chrom., 255:137-149, 1983.

All or a portion of a primer nucleic acid sequence (naturally occurring or synthetic) may be substantially complementary to a target nucleic acid, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are regions of counterpart, target and capture nucleotide sequences 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments, all but one, two, three, four or five nucleotides in the primer are complementary to the hybridized nucleotide sequence in the template.

Primers that are substantially complimentary to a target nucleic acid sequence are also substantially identical to the compliment of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Primer sequences and length may affect hybridization to target nucleic acid sequences. Depending on the degree of mismatch between the primer and target nucleic acid, low, medium or high stringency conditions may be used to effect primer/target annealing. As used herein, the term “stringent conditions” refers to conditions for hybridization and washing. Methods for hybridization reaction temperature condition optimization are known to those of skill in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2× SSC, 0.1% SDS at 50° C. Another example of stringent hybridization conditions are hybridization in 6× sodium chloride/sodium citrate (SSC) at about 45° C., followed by one or more washes in 0.2× SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2× SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

In some embodiments primers can include a nucleotide subsequence that may be complementary to a solid phase nucleic acid primer hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). A primer may contain a nucleotide subsequence not complementary to or not substantially complementary to a solid phase nucleic acid primer hybridization sequence (e.g., at the 3′ or 5′ end of the nucleotide subsequence in the primer complementary to or substantially complementary to the solid phase primer hybridization sequence).

A primer or other sequence of the invention, in certain embodiments, may contain a modification or nucleotide substitution such as inosines, abasic sites, locked nucleic acids, minor groove binders, duplex stabilizers (e.g., acridine, spermidine), or Tm modifiers. In another embodiment, the modification or nucleotide substitution serve to change the binding properties of the primers or probes. In another embodiment, the nucleotide substitution or modification may be a naturally occurring nucleotide (cytosine dideoxynucleotide, guanosine didedeoxynucleotide, adenine dideoxynucleotide, or thymine dideoxynucleotide) that is not present in wild type DNA sequence. A primer, in certain embodiments, may contain a detectable molecule or entity (e.g., a fluorophore, radioisotope, colorimetric agent, particle, enzyme and the like). When desired, the nucleic acid can be modified to include a detectable label using any method known to one of skill in the art. The label may be incorporated as part of the synthesis, or added on prior to using the primer in any of the processes described herein. Incorporation of label may be performed either in liquid phase or on solid phase. In some embodiments the detectable label may be useful for detection of targets. In some embodiments the detectable label may be useful for the quantification target nucleic acids (e.g., determining copy number of a particular sequence or species of nucleic acid). Any detectable label suitable for detection of an interaction or biological activity in a system can be appropriately selected and utilized by the artisan. Examples of detectable labels are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., 125I, 131I, 35S, 31P, 32P, 33P, 14C, 3H, 7Be, 28Mg, 57Co, 65Zn, 67Cu, 68Ge, 82Sr, 83Rb, 95Tc, 96Tc, 103Pd, 109Cd, and 127Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and other cofactors or biomolecules such as digoxigenin, strepdavidin, biotin (e.g., members of a binding pair such as biotin and avidin for example), affinity capture moieties and the like. In some embodiments a primer may be labeled with an affinity capture moiety. Also included in detectable labels are those labels useful for mass modification for detection with mass spectrometry (e.g., matrix-assisted laser desorption ionization (MALDI) mass spectrometry and electrospray (ES) mass spectrometry).

### Multiplexing

Methods provided herein allow for high-throughput detection of nucleic acid species in a plurality of nucleic acids (e.g., nucleotide sequence species, amplified nucleic acid species and detectable products generated from the foregoing). Multiplexing refers to the simultaneous detection of more than one nucleic acid species. General methods for performing multiplexed reactions in conjunction with mass spectrometry, are known (see, e.g., U.S. Pat. Nos. 6,043,031, 5,547,835 and International PCT application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly-automated processes for analyzing sequence variations with high speed and accuracy, in some embodiments. In some embodiments, methods herein may be multiplexed at high levels in a single reaction.

In certain embodiments, the number of nucleic acid species multiplexed include, without limitation, about 1 to about 20 (e.g., about 1, 2, 3, 4, 5, 6, 7, 8, 9, 10, 11, 12, 13, 14, 15, 16, 17, 18, 19, 20). Design methods for achieving resolved mass spectra with multiplexed assays can include primer and oligonucleotide design methods and reaction design methods. For primer and oligonucleotide design in multiplexed assays, the same general guidelines for primer design applies for uniplexed reactions, such as avoiding false priming and primer dimers, only more primers are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da. In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

Methods and kits described herein can be practiced and assembled using one or more sets of primers to detect a specific HPV type, and sometimes two or three sets of primers are utilized. For multiplex methods described herein, about 3 to about 15 or more sets of primers can be utilized (e.g., about 3, 4, 5, 6, 7, 8, 9, 10, 11, 12, 13, 14, 15, sets). A set of primers can include two amplification primers, optionally one extension primer and optionally one competitor nucleic acid.

In some embodiments, without limitation, sequences were adjusted to obtain suitable molecular weight spacing within the mass spectra without undue variation of primer size, which can alter optimal temperatures for PCR. This methodology was used to test the 16 primers, probes and sequences provided in Tables 1A, 1B and 1D. In some embodiments, there is use of no more than 15 contiguous bases, with substitution of the “wild card” base deoxyinosine for deoxyguanosine, deoxyadenine, or deoxythymidine. This concept is derived in relation to the size of the human genome, so that the number of permutations afforded by 16 or more bases (ca. 4.sup.16) is larger than the human genome size. Using such embodiments, it was found that the substitution of an internal deoxyinosine had no effect on PCR conditions or performance of the PCR assay. The primers used for the 16 target sequences are listed in Table 1A-B. Thus, in some embodiments, a stretch of sequence greater than 15 nucleotides was not used, which otherwise has been related to given sequence in the human genome (a sequence must be this long to be represented uniquely in the human genome). Thus, in some embodiments, contiguous sequences used are too small to be represented uniquely in the human genome.

Moreover, in some embodiments, without limitation, desirable molecular weight spacing is achieved by affixing, as desired, spacer molecules on the 5′ end of MassEXTEND primers (e.g., Tables 1B and 1C), the internal primers used for the mass spectroscopic assay approach utilized. Suitable spacer molecules include, without limitation, phosphorylation, C3 spacers, D spacers, amino modifiers C12, spacers 18, and amino modifiers C6 available from Integrated DNA Technologies (Coralville, Iowa). This achieves the desirable spacing of our primer sequences without making major changes in primer length that would affect PCR condition, thus maintaining optimal PCR conditions for all primer sets at uniform conditions to optimize PCR. Taken together, the approaches of using deoxyinosine and modifiers yield a set of primers adapted for this approach, as used in some embodiments.

Without limitation, some embodiments comprise one or more primer sets for one or more of fifteen higher risk forms of human papilloma virus (HPV 16, 18, 31, 33, 35, 39, 45, 51, 52, 56, 58, 59, 66, 68 and 73), and a single copy piece of the human genome corresponding to a non-repeated intron in one of the human (GADPH gene). This adds two higher risk HPV strains, two lower risk types that cause condylomas and a significant fraction of cytological lesions in the anus, and a human genomic DNA probe that supports adequacy of the amount of DNA being analyzed from a specimen. A spacer of 10 nucleotides (5′-ACGTTGGATG-3′ is placed at the 5′ end of one or more forward and/or reverse primer to ensure that these primers were too large to interfere in mass spectroscopy analysis of the unextended primer, the unextended primer plus a cytosine dideoxynucleotide, and the unextended primer plus a guanosine didedeoxynucleotide.

In some embodiments, one or more nucleotide substitutions are introduced to one or more sequences. In a specific embodiment, a deoxyinosine is substituted for deoxythymidine whenever required to break a run of more than 16 nucleotides of contiguous sequence in the left primer, right primer, and/or unextended sequences.

Some embodiments employ a novel strategy that include placing inert molecular weight markers on oligonucleotides as required to generate fragments of different molecular weights. The molecular weight markers used were those made available for 5′-end modification by Integrated DNA Technologies (Coralville, Iowa). These modifiers are shown in Table 1C below. Other modifiers known in the art also may be used.

The following is a description of the 5′ modifications used in certain HPV assays, specifically for HPV SBE (single-base extension) primers, to increase each primer's overall molecular weight in specific increments. These modifications can be used to derive incremental differences in single-stranded DNA beyond that which can be achieved simply using native DNA bases A (deoxyadenosine), C (deoxycytosine), G (deoxyguanosine) and T (deoxythymidine), which have molecular weights (in daltons) of 313.2, 289.2, 329.2, and 304.2 respectively.

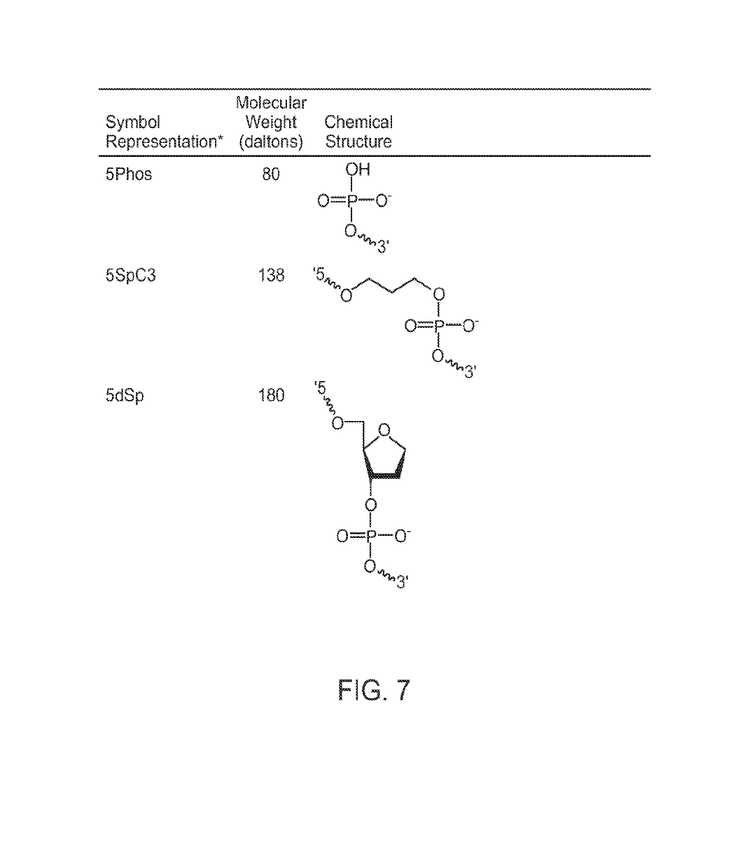

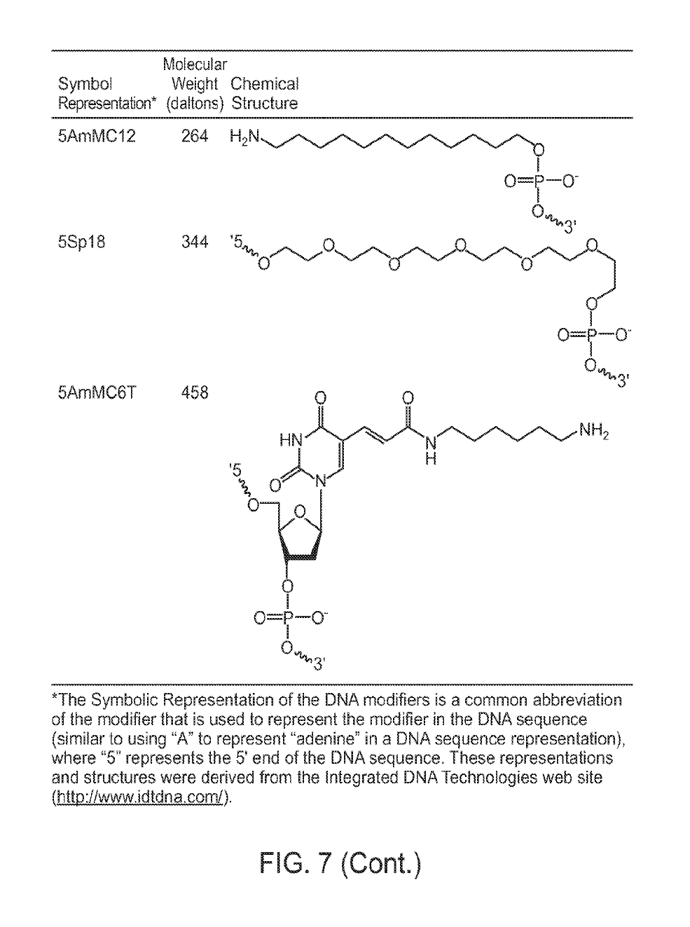

The addition of DNA modifiers (as some examples only, linkers or spacers) to the 5′ end of the primers do not interfere with the performance of the primers, and incrementally changing the overall molecular weight of the primers. 5′ DNA modifiers described in the Examples here have been used to add molecular weight to the primers; however, other suitable modifiers may also be used (See FIG. 7).

Using this strategy some embodiments achieve the separation of 48 peaks, required for the resolution of 16 primer sets by at least 20 daltons (each primer set generates 3 different oligonucleotides: the unextended primer, the unextended primer plus a cytosine dideoxynucleotide and the unextended primer plus a guanosine didedeoxynucleotide).

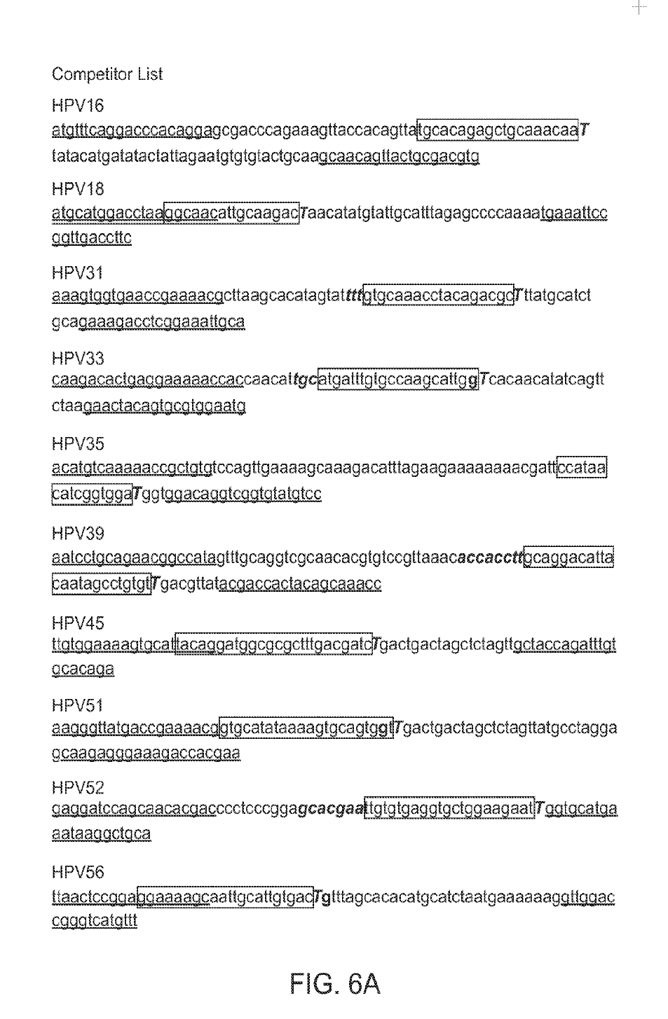

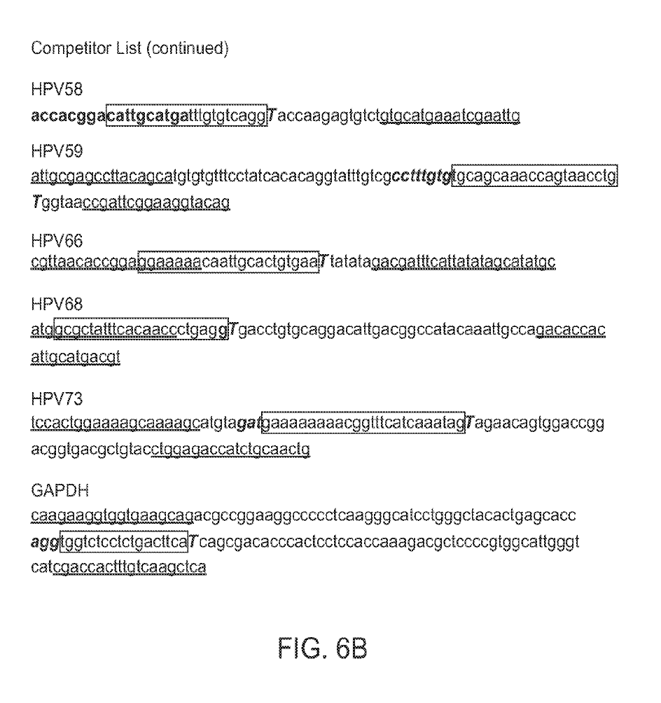

Competitor nucleic acids can be designed and synthesized in a number of manners known in the art. Competitor sequences are provided in FIG. 6. In some embodiments, without limitation, competitor sequences may be scrambled in order to avoid constructing an intact oligonucleotide long enough to hybridize under moderately stringent conditions. To do this, the left, right and unextended primer sequences in the competitor are maintained. However, the sequences between the left primer and unextended primer are inverted and the sequences between the right primer and unextended primer are inverted. This approach maintains the sequence of the left, right and unextended primer sites so that the PCR mass spectroscopy assay proceeded unimpeded. However, by decreasing the shared homology to only the length of the primer sites (18-25 nucleotides rather than to the full 100 nucleotide length of the competitor), this strategy destroyed the capability of the competitor to hybridize to the HPV effectively. This inability to hybridize effectively is because nucleic acid hybridization requires a stretch of homology of ca. 100 nucleotides significantly in excess of 18-25 nucleotides stretch sufficient for priming in PCR). As a result, the scrambled competitor no longer fulfilled the requirement of multiple methods that a 100 nucleotide fragment of HPV would hybridize to a HPV virus under moderately stringent conditions. As a result, the competitors represent novel DNA sequences that do not meet the criteria of retaining homology sufficient to allow hybridization under moderately stringent conditions.

This approach allows the extension in some embodiments to 15 high risk HPV types. In some embodiments, a method can include a human genomic DNA probe to accomplish detection of the amount of human genomic DNA put into the reaction. As a result, the approach supports the quantification of the copy number of each HPV type/cell, making some embodiments quantitative by themselves without requiring a separate assay. The approach provides novel sequences for left primer, right primer, unextended primer and competitors.

In some embodiments, the sequences were chosen so there was no molecular weight overlap <ca. 20nt between the sequences corresponding to the unextended primer, the wild type gene, and the internal competitor for each of the 15 different types of HPV. In all, such a system may discriminate each of the 3.times.15+2.timesGAPDH=47 different peaks (the peaks distinguished by mass spectrometry were unextended primer; unextended primer+wild type gene sequence (unextended primer plus either a C or G, depending on the next nucleotide of the gene); and unextended primer+internal competitor sequence (unextended primer plus either a G or C, depending on the next nucleotide of the competitor). These distinctions were based on the ability of the mass spectrometry-based method to distinguish a separation of ca. 20 daltons between 2 molecular weights.

FIG. 2 depicts profile results of a mass spectroscopic assay screen in accordance with some embodiments for 15 high risk types of HPV that are screened for in a commercial test (i.e., HPV 16, HPV 18, HPV 31, HPV 33, HPV 35, HPV 39, HPV 45, HPV 51, HPV 52, HPV 56, HPV 58, HPV 59, HPV 66, HPV 68 and HPV 73). The 15 different peaks corresponding to the molecular weights of the MassEXTEND primer for each of the 15 distinct high-risk HPV types (HPV 16, 18, 31, 33, 35, 39, 45, 51, 52, 56, 58, 59, 66, 68 and 73) are shown. The lines without peaks denote where the MassEXTEND competitors and gene products would map (representing a potential total of 3.times.13=39 non-overlapping peaks; for simplicity, only the 15 unextended peaks are shown).

FIG. 2 illustrates the ability to detect and distinguish a variety of HPV DNA sequences in some embodiments. In some embodiments, an appropriate set of outside primers and an appropriate unextended primer MassExtend sequence (ca. 20 nt) for each ca. 100 nt HPV E6 sequence can be used for the genomic DNA standard, for Chlamydia trachomatis, and for Neisseria gonorrhoea. An oligonucleotide corresponding to each of the ca. 100 nt sequences was synthesized, with one base changed (a C for a G, or a G for a C). The synthesis was done, for example, using a commercially available oligonucleotide synthesizer (e.g., service afforded by Integrated DNA Technologies (IDT)). Ca. 100 nt long oligonucleotides were synthesized using sequences corresponding to the internal competitor sequence for each of the 15 different types of HPV, the genomic DNA standard, Chlamydia trachomatis, and Neisseria gonorrhoea. For each of the 18 sequences, ca. 20 nt primers (to which tags were added to eliminate interference with the mass spectroscopic profile shown in FIG. 2) were synthesized corresponding to the right and left ends of these ca. 100 nt long oligonucleotides. Finally, a mass spectroscopic assay extension primer was synthesized, comprising a sequence directly abutting a C or G (in which case the internal competitor resulted in the incorporation of a G or C, respectively, such that it was possible to distinguish the wild type gene sequence from the internal competitor sequence) using this one nucleotide difference.

In some embodiments, the primer sequences may be identical for the wild type gene sequence and internal competitor. The only difference between the wild type gene sequence and internal competitor is the one nucleotide adjacent to the unextended primer sequence. Given this identity of sequence, both the wild type gene sequence and the internal competitor amplify with the same efficiency. As a result, amplification of a known amount of the internal competitor can be used in some embodiments to quantify the amount of the wild type gene sequence that is amplified. In some embodiments, the primer extension rate for a particular HPV type is greater than about 30%, 35%, 40%, 45%, 50%, 55%, 60%, 65%, 70%, 75%, 80%, 85%, 90% or 95% or more. In certain embodiments, the primer extension rate for all of the HPV assays is greater than about 15%, 20%, 25%, 30%, 35%, 40%, 45%, 50%, 55%, 60%, 65%, 70%, 75%, 80%, 85%, 90% or 95% or more.

In some embodiments, without limitation, the unextended primers, unextended primers+guanosine and the unextended primers+cytosine (3.times.22 primers=66 total primers) should all fit in a molecular weight space between about 5000 and about 8500 daltons, and be separated by a minimum distance of ca. 20 daltons. At the same time, the length of the primers are constrained by the requirement that they bind and function as templates within a small temperature range so that they will all yield amplification at the same temperature. To accomplish these goals, a novel strategy of affixing various inert spacer molecules to the 5′ end of the unextended primer was developed. These molecules are also referred to as mass modifiers.

For some embodiments, without limitation, amplification primers that can be used for the first PCR amplification are provided in Table 1A. In some embodiments, primers that can be used for primer-mediated extension are provided in Table 1B. In certain embodiments, sequences of competitors that can be used are provided in FIG. 6 and Table 1D. The spacers that can be used are detailed in Table 1C. Primer sequences also are shown for HPV types 16, 18, 23, 26, 31, 33, 35, 39, 45, 51, 52, 53, 56, 58, 59, 66, 68, and 73. A measure of total genomic DNA input using a standard control, for example, an intron of the gene erbB-2, may be included as part of the assay. In some embodiments, primers and probes for the diagnosis of other sexually transmitted diseases may be included. For example, infections of gynecological import (Chlamydia trachomatis and Neisseria gonorrhoeae) may also be included. Some embodiments comprise screening simultaneously for 15 types of HPV, a measure of total genomic DNA and tests for other infection(s), such as Chlamydia trachomatis and Neisseria gonorrhoeae.

Methods and compositions described herein may be utilized in conjunction with other assays and can be utilized to determine the type and amount of HPV present in serum and/or blood, including but not limited to, due to tumorigenesis. Since the technique of screening serum and/or blood is maximally sensitive when screening the HPV probe of choice, the screen of tumor and/or ThinPreps may be used to determine whether HPV is present, and if so, which type of HPV. That type of HPV may then be used to screen blood and/or serum with maximum efficiency; if several types of HPV are present, each type can be screened for individually. In some embodiments, the presence of HPV in tumor or ThinPreps is at concentrations higher than in serum and/or blood.

As discussed above, the certain commercial test do not reveal which HPV type is found in the cervix ThinPrep. Without limitation, some embodiments described here address shortcomings of a target-capture-based method by:

1. including an application of the multiple capabilities of the mass spectroscopic assay screen;
2. accurate diagnosis without cross-reaction from related HPV sequences occurs because the molecular weight of each HPV type-specific reaction product is accurate to .+−.ca. 20 daltons, so it is specific for the sequence of a given HPV type. In fact, the mass spectroscopic assay test of some embodiments distinguishes each of the 15 high risk types of HPV detectable by a commercial screen without cross-reaction with other HPV viruses (FIG. 2);
3. The mass spectroscopic assay of some embodiments is positive down to the level of individual molecules (at which level one may see expected Poissonian variation); and
4. Current methods that detect cervical disease rely on two major technologies: (i) detection of cytological anomalies of exfoliated cervical cells, the ‘Pap’ smear developed by Dr. G. N. Papanicolaou; and (ii) detection of HPV infection. Drawbacks of cytology are inter-observer reliability, limited sensitivity (.ltoreq.85%) and reliance on highly-trained individuals to perform tests. The sensitivity of Pap smears is considered adequate for clinical purposes only by repetitive screening. Consequently, the loss of individuals to regular follow-up and the inability of even repeated uses of the cytological Pap test to detect all individuals with cervical abnormalities contribute to cervical cancer incidence in screened populations.

An alternative to cytologic methods may be used to accomplish direct detection of HPV, a cause of virtually all cervical carcinomas. HPV currently is detected by an FDA-approved HC2 Test™ (Qiagen Corporation, Bedford, Mass.), that uses a cocktail of type-specific hybridization probes to detect 13 types of high-risk HPV associated with cervical malignancies PCR using degenerate oligonucleotides or a suite of diagnostic tests that detects and then types the form of HPV that is present (Roche). Drawbacks to these methods are limited sensitivity, specificity and quantitative abilities. Sensitivity is limited as ca. 10.sup.2-10.sup.3 molecules are required to be detected by these tests. Specificity is limited due to cross reaction of HC2 with non-high-risk strains of HPV. ca. 10% of the time due to cross reaction with non-high-risk strains of HPV. In addition, the HC2 test does not permit accurate quantification.

Accordingly, provided herein are systems, compositions, and methods that accomplish detection at a very sensitive level, which enables observations that were not previously possible. Without limitation, some embodiments take advantage of the finding that small amounts of HPV in body fluids are associated with cancer or dysplasia, which can then be eliminated by removal of the tumor or dysplasia. Thus, in certain embodiments, methods include (a) treating a subject having a condition associated with HPV infection (e.g., cancer, dysplasia; surgical, radiation, chemotherapy treatment), and (b) detecting the presence, absence and/or amount of the HPV associated with the condition before, during or after step (a).

### Kits

Kits often comprise one or more containers that contain one or more components described herein. A kit comprises one or more components in any number of separate containers, packets, tubes, vials, multiwell plates and the like, or components may be combined in various combinations in such containers.

One or more of the following components, for example, may be included in a kit: (i) one or more amplification primers described herein, (ii) one or more extension primers described herein, (iii) a solid support for multiplex detection (e.g., detection of amplification products and/or extended extension primers; a solid support that includes matrix for matrix-assisted laser desorption ionization (MALDI) mass spectrometry; (iv) reagents for detecting amplification products and/or extension products; (vi) a detector for detecting amplification products and/or extension products (e.g., mass spectrometer); (vii) reagents and/or equipment for isolating a tissue, fluid, cell and/or nucleic acid sample from a subject; (viii) one or more competitor nucleic acids; (ix) software and/or a machine for analyzing signals resulting from a process for detecting amplification products and/or extension products; (x) information for identifying presence, absence and/or amount of an HPV type (e.g., a table or file that converts signal information to a call), (xi) reagents for isolating nucleic acid (e.g., DNA, RNA) from plasma, serum, urine or other fluid, tissue or cells; (xiv) reagents for stabilizing serum, plasma, urine, or other fluid or cells, or nucleic acid for shipment and/or processing.

A kit sometimes is utilized in conjunction with a process or composition described herein, and can include instructions for performing one or more processes and/or a description of one or more compositions. Instructions and/or descriptions may be in tangible form (e.g., paper and the like) or electronic form (e.g., computer readable file on a tangle medium (e.g., compact disc) and the like) and may be included in a kit insert. A kit also may include a written description of an internet location that provides such instructions or descriptions (e.g., a URL for the World-Wide Web). Thus, provided herein is a kit that comprises one or more (a) amplification primers (e.g., Table 1A), (b) extension primers (e.g., Table 1B) and/or (c) competitor nucleic acids (e.g., Table 1D). A kit also comprises a conversion table, software, executable instructions and/or an internet location that provides the foregoing, in certain embodiments, where a conversion table, software and/or executable instructions can be utilized to convert detection data into a call of the presence, absence and/or amount of an HPV type, for example. In some embodiments, a kit comprises reagents and/or components for performing an amplification reaction or extension reaction (e.g., polymerase, nucleotides, buffer solution, thermocycler, oil for generating an emulsion).

### Examples

The examples set forth below illustrate certain embodiments and do not limit the technology.

In some embodiments, the invention comprises, without limitation, the use of matrix-assisted laser desorption ionization-time of flight (“MALDI-TOF”) mass spectrometry (“MS”) for qualitative and quantitative gene expression analysis in combination with aspects of competitive PCR, primer extension reaction, and MALDI-TOF MS (see generally FIG. 1). A sample thought to contain HPV nucleic acid (e.g., DNA or RNA) isolated from a biological sample is spiked with a synthetic oligonucleotide (the competitor) with a sequence identical to or substantially matching a portion of the nucleic acid sequence of an HPV of interest except for one or more mismatch(es), for example, one single base roughly in the middle of the sequence of interest. In some embodiments, the competitor is added in known concentration (for example, at a concentration of 250 attoMolar). The competitor and the nucleic acid of interest are co-amplified by PCR in the presence of forward and reverse primers. Excess dNTPs and primers are removed by means known to those of ordinary skill after PCR, as one example only and without limitation, enzymatic digestion and appropriate washing. Then, a base extension reaction is carried out with an extension primer and a combination of different ddNTPs (as one example only, G and C). The extension primer hybridizes right next to the single base extension site and at least one of two ddNTP bases is added differentially for the competitor and the DNA, yielding two oligonucleotide products with different molecular weights. In a typical molecular weight window of about 5,000 to about 8,500 Daltons (Da), the MALDI-TOF MS easily distinguishes two oligonucleotides if they differ by more than ca. 20 Da. In accordance with some embodiments, these differential extension products are identified qualitatively, and their concentrations can be quantified in relation to their ratio from the MALDI-TOF MS, as one example only, when the concentration of the added competitor sequence is known. In some embodiments, without limitation, desirable molecular weight spacing is further achieved by affixing, as desired, spacer molecules on the 5′ end of the base extension primers, as described further herein.

Preparation and Quantification of Nucleic Acid from Samples.

Cervical samples were collected in ThinPrep PreservCyt solution (Hologic Corporation, Bedford, Mass.). Following reporting of patient results, specimens were unlinked to patient identifiers, and aliquots were prepared and tested by mass spectroscopic PCR methods described hereafter. The DNA was isolated from about 5 ml of ThinPrep solution by rotating with about 10.mu.l of Zymo beads from the ZR Serum DNA Isolation kit. The beads were added to the sample and about 4 times the volume of Genomic Lysis Buffer (Zymo Research Corporation) was added. The mixture was tumbled overnight at about 4.degree. C. DNA was prepared from the beads according to the manufacturer's instructions. Final suspension was in a small volume (about 20.mu.l) of Elution Buffer. Samples were run for Qiagen HC2 and Roche analyses (including reverse line blotting) according to the manufacturers' instructions. Samples were then provided blindly for mass spectroscopic analysis.

In another embodiment, RNA is isolated from the biological sample to be tested. RNA may be isolated by any method known in the art. For example, RNA extraction methods for use in methods described herein include the following commercially available extraction methods suitable for extraction of intracellular, extracellular and/or viral RNA: TRIzol™ (Life Technologies); Trisolv™ (BioTecx Laboratories); ISOGEN™ (Nippon Gene); RNA Stat™ (Tel-test); TRI Reagent™ (Sigma); SV Total RNA Isolation System (Promega); RNeasy Mini Kit, QIAamp MinElute Virus Spin or QIAamp MinELute Virus Vacuum Systems (Qiagen, Hilden, Germany); Perfect RNA: Total RNA Isolation Kit (Five Prime-Three Prime Inc., Boulder, Colo.); or similar commercially available kit, wherein extraction of RNA may be performed according to manufacturer's directions, or adapted to the biological sample believed to comprise HPV nucleic acid. Prior to detection, the specific HPV RNA's may be subjected to some form of a nucleic acid amplification assay to the extracted RNA, whereby the extracted RNA may first be reverse transcribed to cDNA prior to amplification of the cDNA.

### Mass Spectroscopic Methods of Analyzing HPV.

In accordance with some embodiments, without limitation, a multi-step process of real-time competitive PCR (rcPCR), primer extension and MALDI-TOF MS separation of products on a matrix-loaded silicon chip array is used to detect as few as several initial molecules. A competitive nucleotide template (as one example only, ca. 100 nt) is synthesized to match an HPV target sequence for PCR except for a single base mutation in the competitor, which is introduced during the synthesis. The single base change can then be discriminated from the HPV target allele using a primer extension reaction with product resolution by mass (in Daltons) on the MALDI-TOF MS as is done analogously for SNP genotyping. In some embodiments, the competitive template is added to the PCR reaction at known quantities and can be titrated to create a standard curve for the determination of target DNA quantities. When the peak areas of the target allele and competitive template allele are equal, the concentrations of the two molecules are at about a 1:1 ratio, representing the amount of target DNA in the reaction. The mass spectroscopic analysis is very specific as, in this exemplary embodiment, a given primer extension product was discerned down to a resolution of ca. 20 daltons. Any contaminant products would therefore have to be this specific size to generate a false-positive signal. The presence of the internal standard (competitive template) also serves to confirm that the enzymes required for PCR were working and that the sample was purified free of inhibitors of PCR.

In accordance with some embodiments, without limitation, a 16-plex HPV assay was designed by first deriving PCR and extension primer sequences with Primer3 software (http://frodo.wi.mit.edu/cqi-bin/primer3/primer3_www.cgi) from the E6 region of the various HPV strains. These sequences were then used to define input sequence boundaries for use with MassARRAY assay designer software v3.0. (Sequenom, Inc., San Diego, Calif.). In this manner, each of the 15 discrete types of high-risk HPV were distinguished (FIG. 1). Forward and reverse primer, extension primer, and competitor sequences are disclosed in Table 1A-B and 1D, respectively. Additional considerations were used to formulate the final designs. These included the placement of sequence variations of HPV isolates reported in the literature. Avoidance of PCR or extension primers which included the known variants was done by referring to sources including: Human Papillomaviruses 1997 Compendium; Steinau et al, Journal of General Virology 2010, Wu et al, J Gen Virol. 2006 May; 87(Pt 5):1181-8; and other public sources. Consideration was also made of splice sites in the high risk transcript E6*I, which was first described by Tang et al. J Virol. 2006 May; 80(9): 4249-4263. This transcript is strongly related to disease progression, and is considered a key feature of high risk viruses. Additional clarification and fine mapping of the transcript in all high risk types is available in the following: (Ren et al, (2009) April; 23(2):88-90) and (Sotlar et al., Journal of Medical Virology 74:107-116 (2004)).

Conditions for multiplexed rcPCR mass spectroscopic analysis of PCR have been described previously. Reactions were initiated by generating a 96 well master plate from which a 384 well reaction plate was established using an EPmotion robot. There were 4 wells at 50 aM (attomolar=10.sup.-18 M) of a given competitor for each HPV type. This amount of competitor inhibits carry-over contamination during processing, and can be adjusted to reflect an amount which suppresses low, non clinically relevant infections. Clinical relevance has been established by many studies at 10,000 copies in 2 mL of ThinPrep sample.

Because MassARRAY is not a homogeneous assay, attention should be paid to setting up the reaction. Two robots were utilized (before and after the initial PCR) to set up reactions and minimize contamination. The routine control in every plate showing that normal samples were negative confirmed that these techniques to prevent contamination were effective. All values reported herein represent the analysis of at least 4 independent data points.

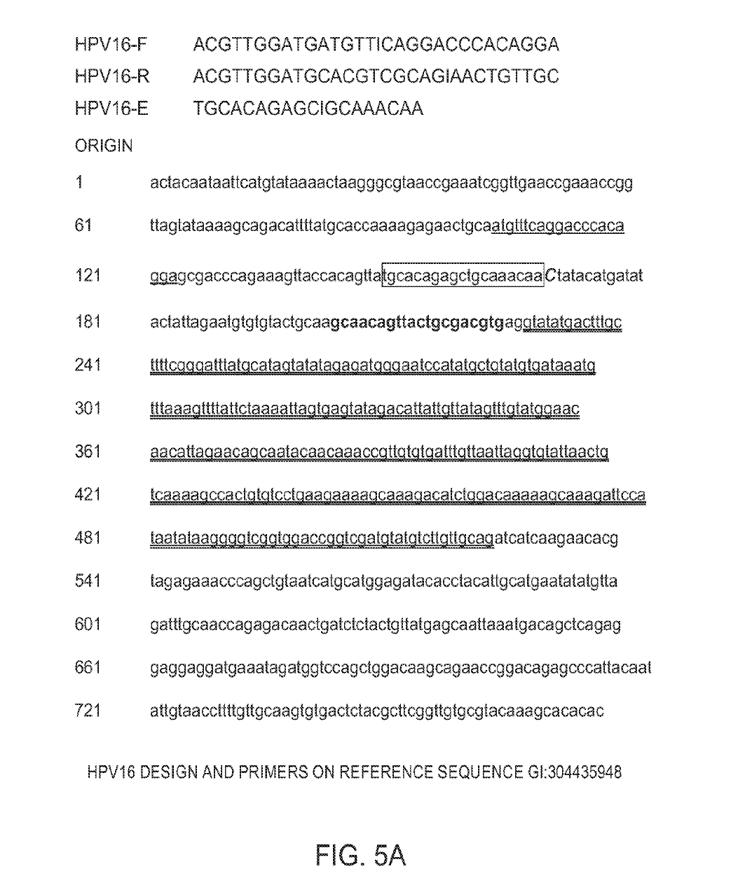

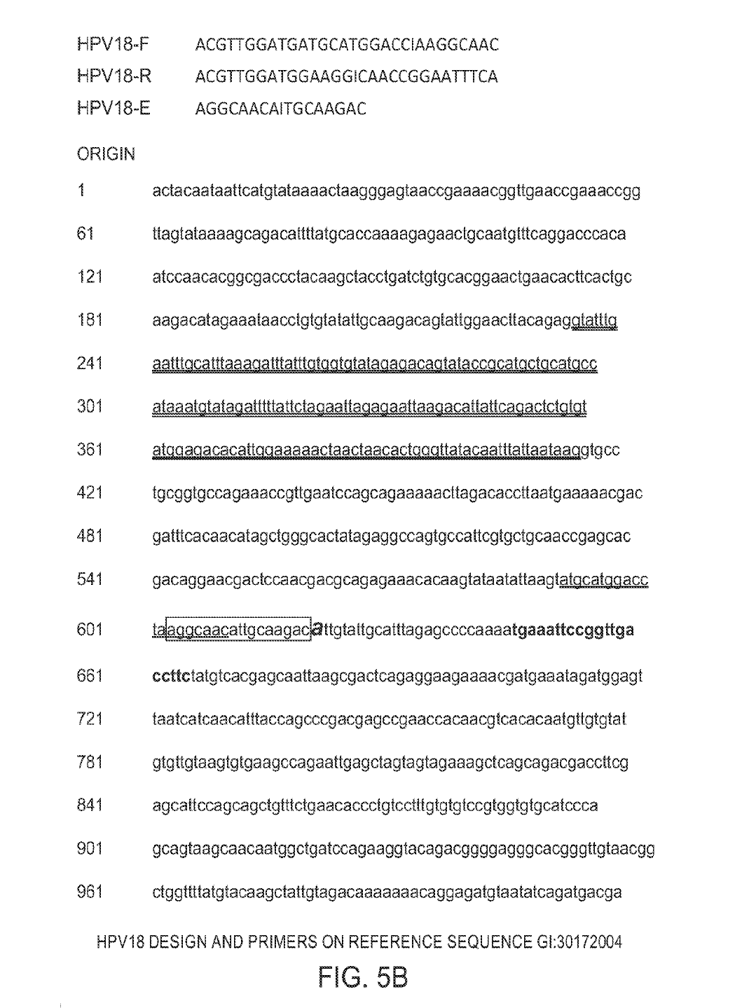

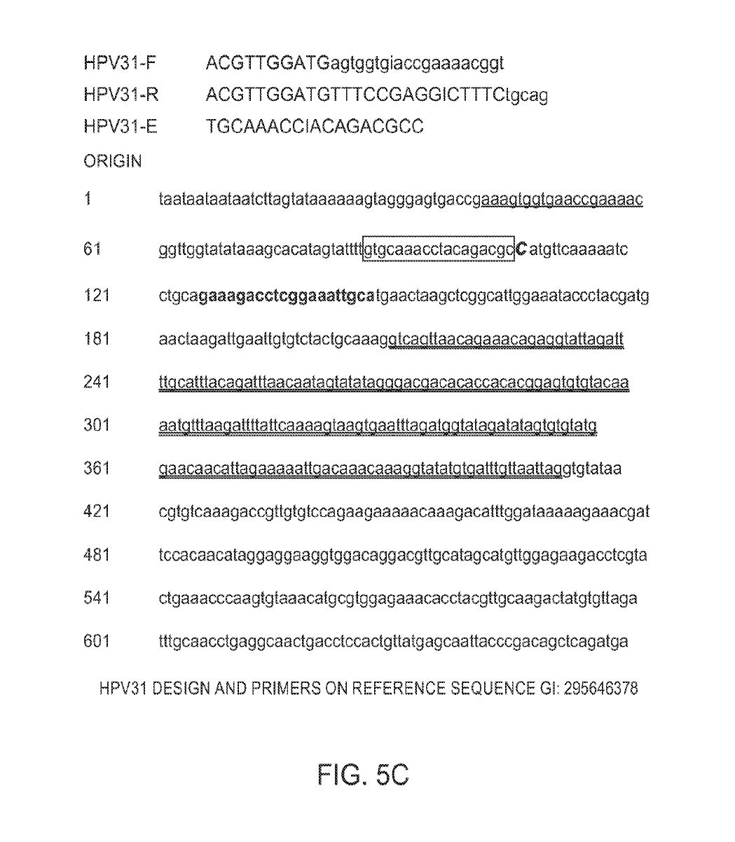

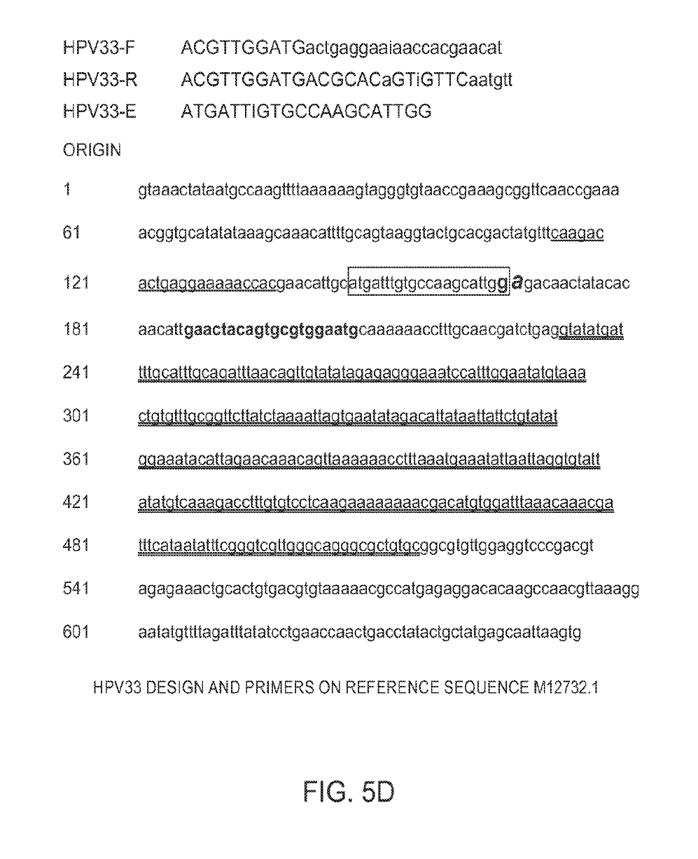

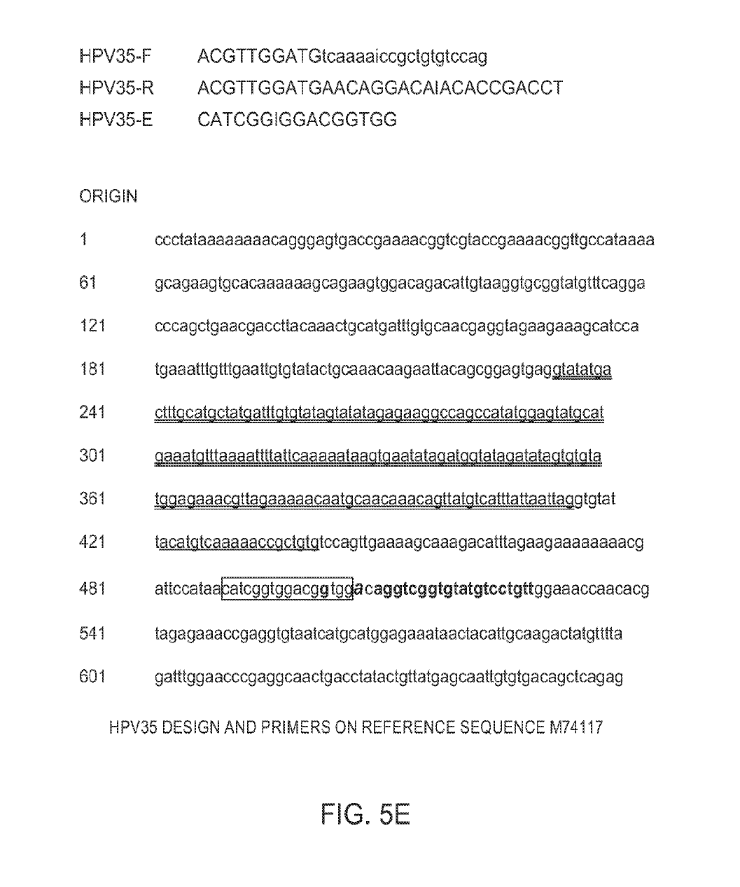

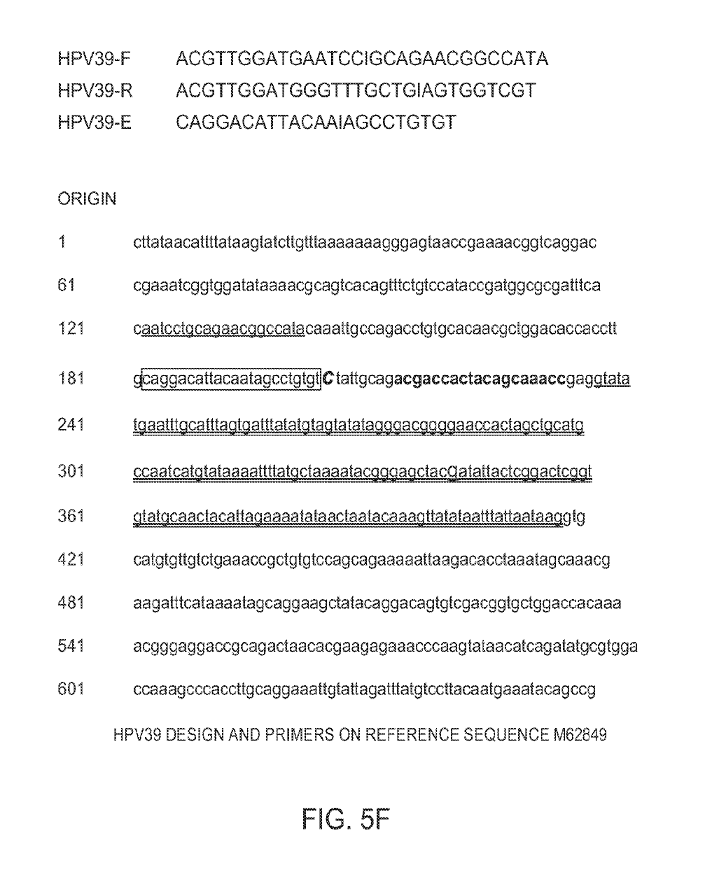

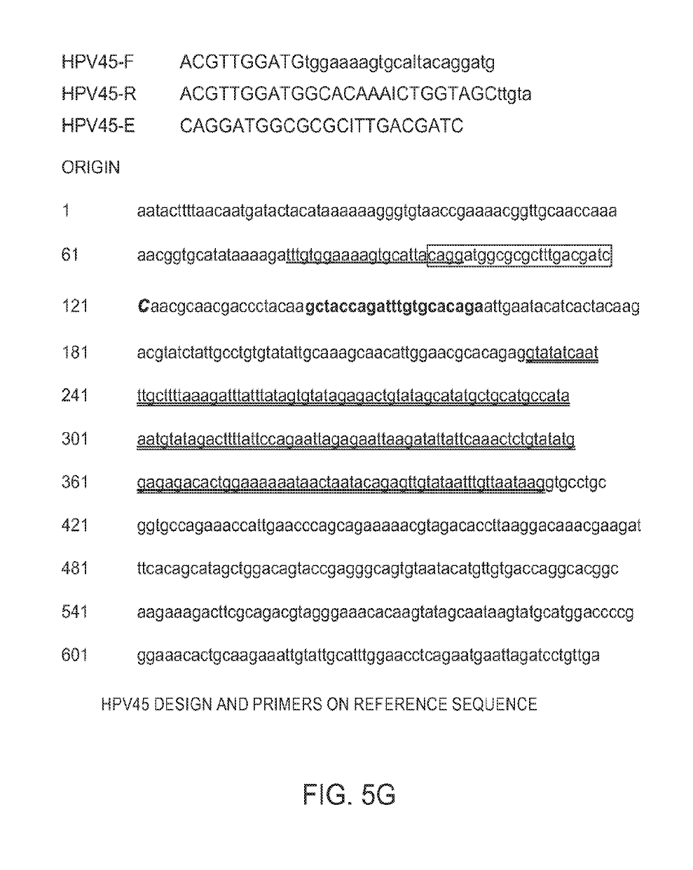

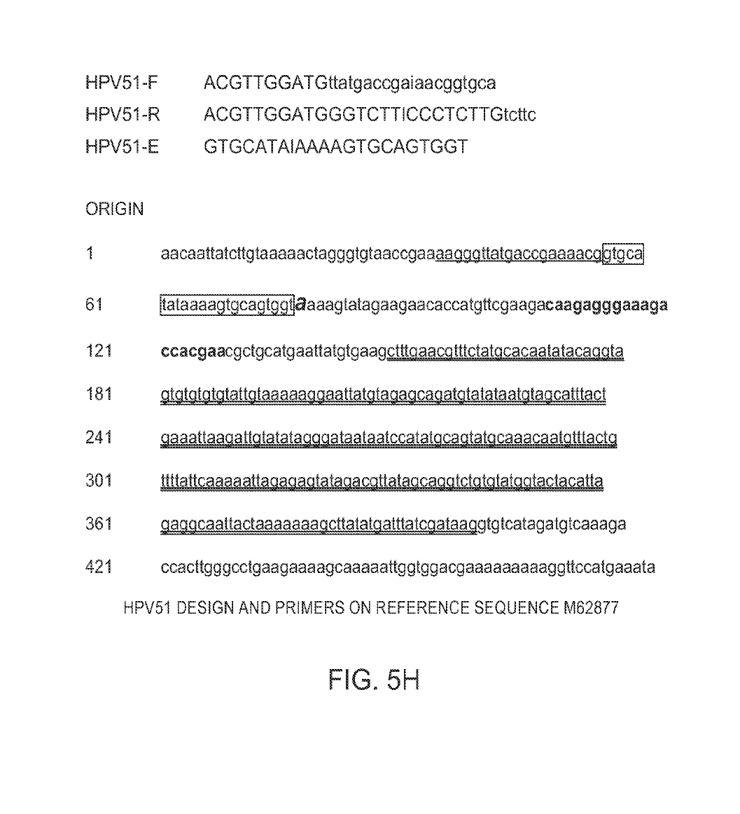

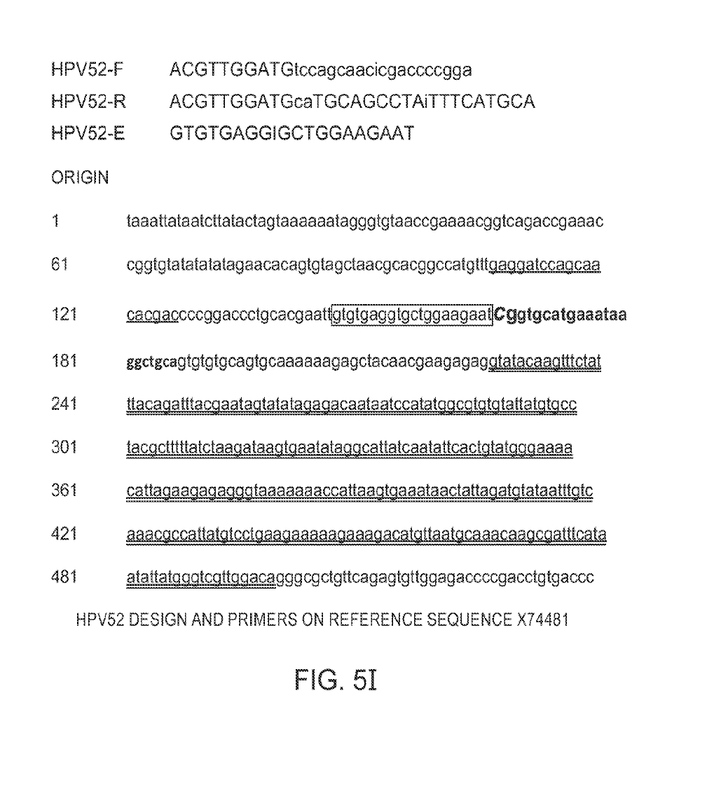

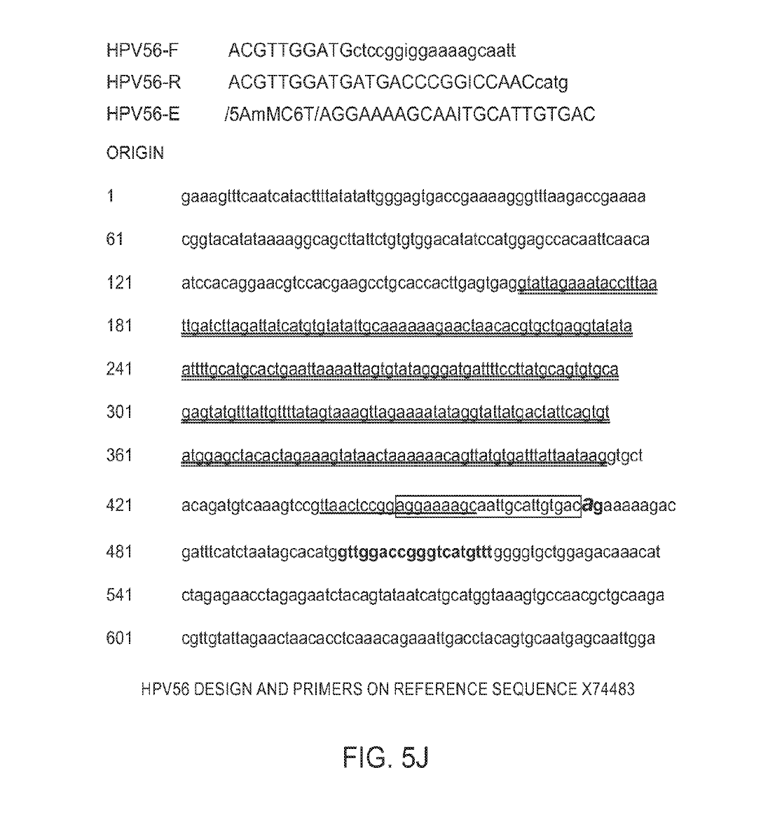

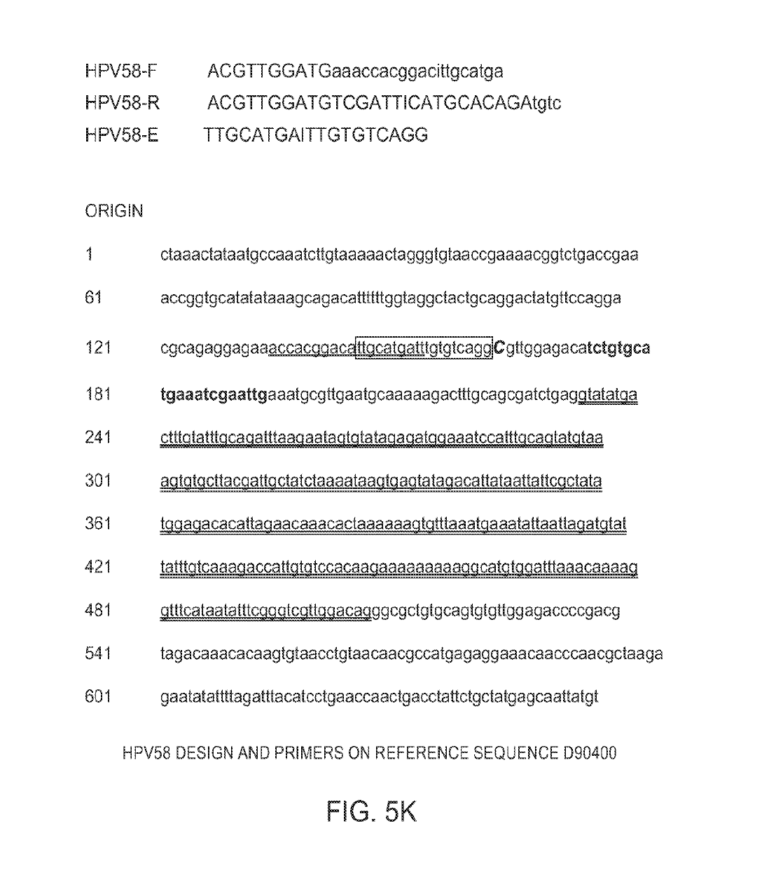

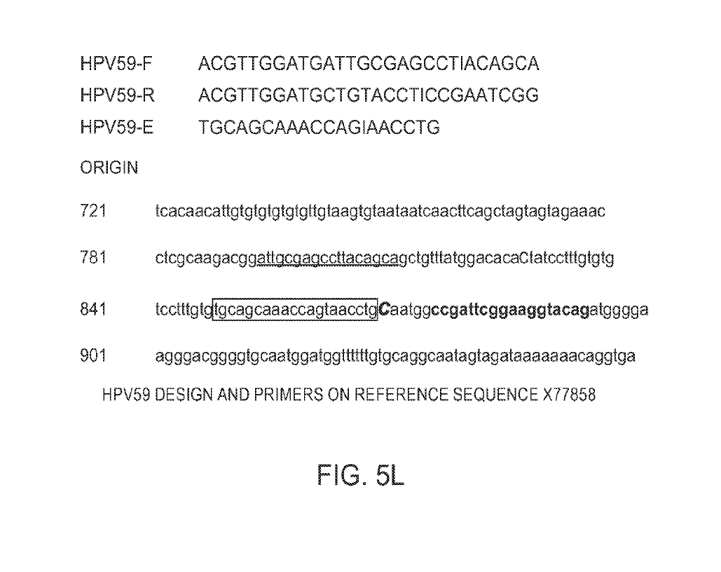

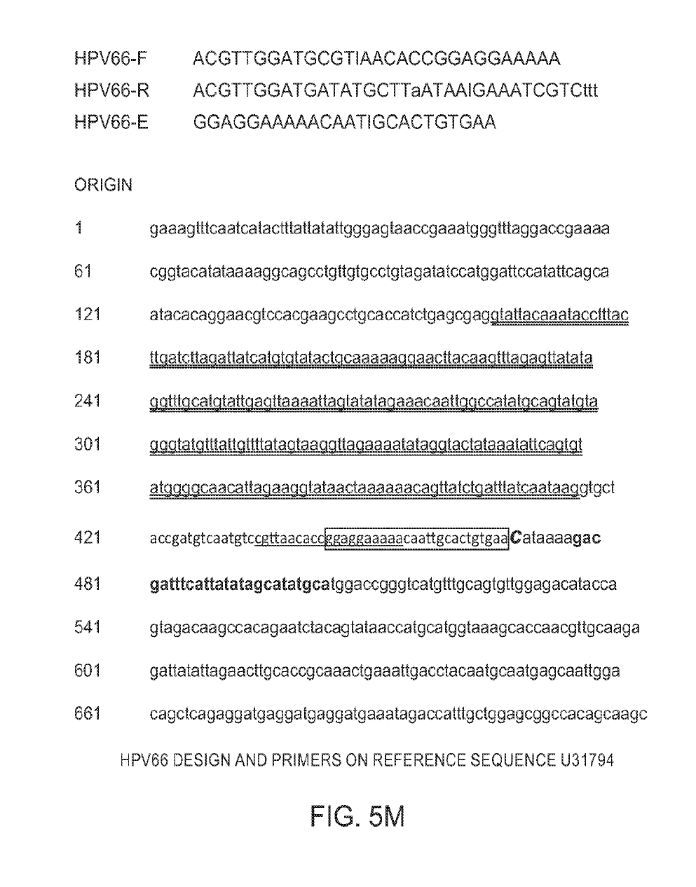

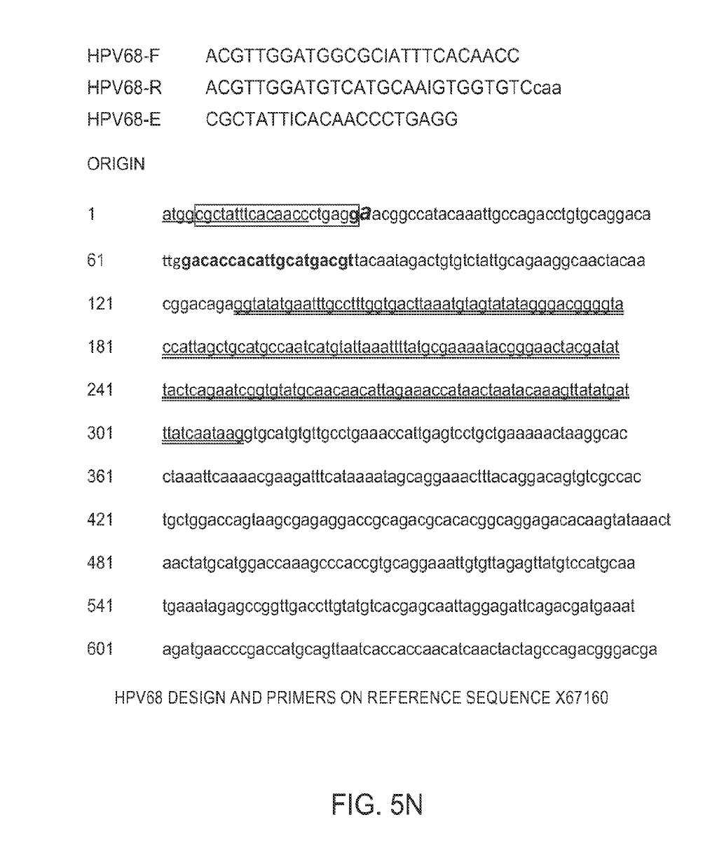

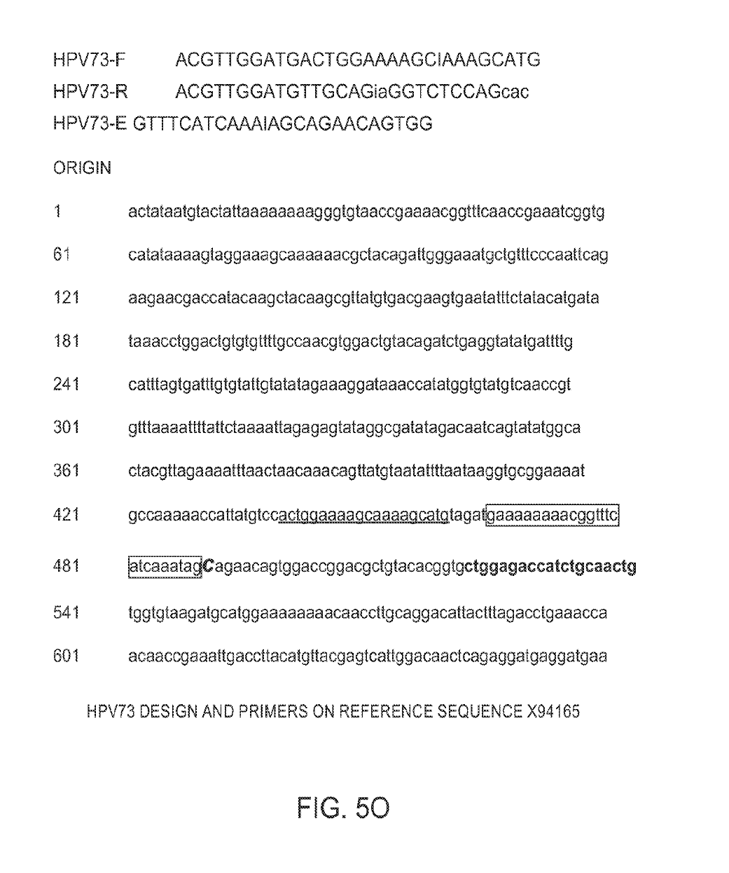

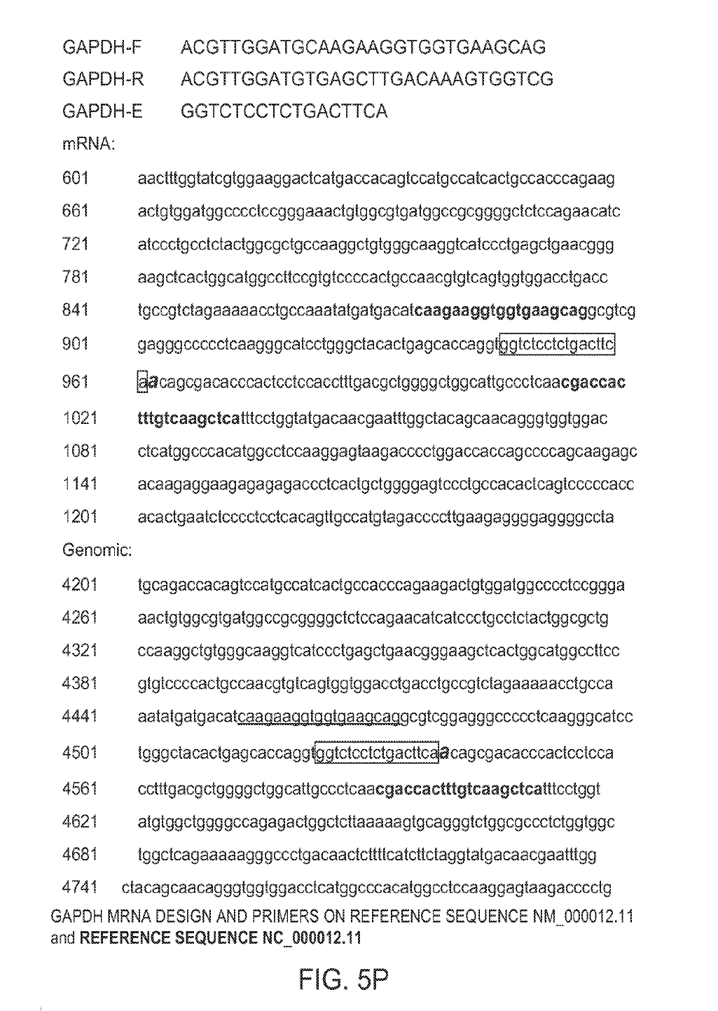

By way of additional example only, without limiting embodiments herein, in a first stage, tumors or cervical ThinPreps were screened for one of the 15 high risk types, using an assay embodiment to identify separately the presence or absence of the 15 different types of high risk HPV in a single reaction. Primer sequences were derived from the E6 region of each of the 15 types of HPV that are high risk for human cancer. See Tables 1A-1C and FIGS. 5A-5P.

TABLE 1A
PCR Primer Sequences

| | | Primer | | | Primer |
|---|---|---|---|---|---|
| FORWARD | PRIMER SEQUENCE | Length | REVERSE | PRIMER SEQUENCE | Length |
| HPV16 | ACGTTGGATGATGTTICAGGACCCACAG | 30 | HPV16 | ACGTTGGATGCACGTCGCAGIAACT | 30 |
| | GA | | | GTTGC | |
| | (SEQ ID NO 1) | | | (SEQ ID NO 16) | |
| HPV18 | ACGTTGGATGATGCATGGACCIAAGGC | 30 | HPV18 | ACGTTGGATGGAAGGICAACCGGAA | 30 |
| | AAC | | | TTTCA | |
| | (SEQ ID NO 2) | | | (SEQ ID NO 17) | |
| HPV31 | ACGTTGGATGAAAGTGGTGAICCGAAAA | 30 | HPV31 | ACGTTGGATGTTTCCGAGGICTTTCT | 30 |
| | CG | | | GCAG | |
| | (SEQ ID NO 3) | | | (SEQ ID NO 18) | |
| HPV33 | ACGTTGGATGCAAGACACIGAGGAAAA | 32 | HPV33 | ACGTTGGATGCATTCCACGCACIGT | 30 |
| | ACCAC | | | AGTTC | |
| | (SEQ ID NO 4) | | | (SEQ ID NO 19) | |
| HPV35 | ACGTTGGATGACATGTCAAIAACCGCTG | 30 | HPV35 | ACGTTGGATGAACAGGACAIACACC | 30 |
| | TG | | | GACCT | |
| | (SEQ ID NO 5) | | | (SEQ ID NO 20) | |
| HPV39 | ACGTTGGATGAATCCIGCAGAACGGCC | 30 | HPV39 | ACGTTGGATGGGTTTGCTGIAGTGG | 29 |
| | ATA | | | TCGT | |
| | (SEQ ID NO 6) | | | (SEQ ID NO 21) | |
| HPV45 | ACGTTGGATGTTGTGGAAAAGIGCATTA | 32 | HPV45 | ACGTTGGATGTCTGTGCACAAAICT | 31 |
| | CAGG | | | GGTAGC | |
| | (SEQ ID NO 7) | | | (SEQ ID NO 22) | |
| HPV51 | ACGTTGGATGAAGGGTTAIGACCGAAAA | 30 | HPV51 | ACGTTGGATGTTCGTGGTCITTCCCT | 30 |
| | CG | | | CTTG | |
| | (SEQ ID NO 8) | | | (SEQ ID NO 23) | |
| HPV52 | ACGTTGGATGGAGGATCCIGCAACACG | 29 | HPV52 | ACGTTGGATGTGCAGCCTIATTTCAT | 30 |
| | AC | | | GCAC | |
| | (SEQ ID NO 9) | | | (SEQ ID NO 24) | |
| HPV56 | ACGTTGGATGTTAACTCCGGIGGAAAAG | 29 | HPV56 | ACGTTGGATGAAACAIGACCCGGTC | 29 |
| | C | | | CAAC | |
| | (SEQ ID NO 10) | | | (SEQ ID NO 25) | |
| HPV58 | ACGTTGGATGACCACGGACAITGCATG | 30 | HPV58 | ACGTTGGATGCAATTCGATTICATGC | 31 |
| | ATT | | | ACAGA | |
| | (SEQ ID NO 11) | | | (SEQ ID NO 26) | |
| HPV59 | ACGTTGGATGATTGCGAGCCTIACAGCA | 28 | HPV59 | ACGTTGGATGCTGTACCTICCGAAT | 28 |
| | (SEQ ID NO 12) | | | CGG | |
| | | | | (SEQ ID NO 27) | |
| HPV66 | ACGTTGGATGCGTIAACACCGGAGGAA | 30 | HPV66 | ACGTTGGATGTGCATATGCTAIATAA | 36 |
| | AAA | | | TGAAATCGTC (SEQ ID NO 28) | |
| | (SEQ ID NO 13) | | | | |
| HPV68 | ACGTTGGATGAATGGCGCIATTTCACAA | 30 | HPV68 | ACGTTGGATGACGTCAIGCAATGTG | 30 |
| | CC | | | GTGTC | |
| | (SEQ ID NO 14) | | | (SEQ ID NO 29) | |
| HPV73 | ACGTTGGATGTCCACTGGAIAAGCAAAA | 30 | HPV73 | ACGTTGGATGCAGTTGCAGAIGGTC | 30 |
| | GC | | | TCCAG | |
| | (SEQ ID NO 15) | | | (SEQ ID NO 30) | |

TABLE 1B
Extension Primer Sequences

| | | | | Target |
|---|---|---|---|---|
| | Primers | Sequence | Bases | SBE |
| 1 | HPV18-E | AGGCAACAITGCAAGAC (SEQ ID NO. 31) | 17 | A |
| 2 | HPV45-E | CAGGATGGCGCGCITTGACGATC (SEQ ID NO. 32) | 23 | C |
| 3 | HPV39-E | CAGGACATTACAAIAGCCTGTGT (SEQ ID NO. 33) | 23 | C |
| 4 | HPV59-E | TGCAGCAAACCAGIAACCTG (SEQ ID NO. 34) | 20 | C |
| 5 | HPV56-E | /5AmMC6/GAAAAGCAAITGCATTGTGAC (SEQ ID NO. 35) | 21 | A |
| 6 | GAPDH-E | GGTCTCCTCTGACTTCA (SEQ ID NO. 36) | 17 | A |
| 7 | HPV51-E | GTGCATAIAAAAGTGCAGTGGT (SEQ ID NO. 37) | 22 | A |
| 8 | HPV31-E | TGCAAACCIACAGACGCC (SEQ ID NO. 38) | 18 | A |
| 9 | HPV35-E | CATCGGIGGACGGTGG (SEQ ID NO. 39) | 16 | A |
| 10 | HPV33-E | ATGATTIGTGCCAAGCATTGG (SEQ ID NO. 40) | 21 | A |
| 11 | HPV58-E | /5AmMC6/ACATTGCATGAITTGTGTC (SEQ ID NO. 41) | 19 | A |
| 12 | HPV52-E | GTGTGAGGIGCTGGAAGAAT (SEQ ID NO. 42) | 20 | C |
| 13 | HPV73-E | GAAAAAAAACGGITTCATCAAATAGC (SEQ ID NO. 43) | 26 | A |
| 14 | HPV16-E | TGCACAGAGCIGCAAACAA (SEQ ID NO. 44) | 19 | C |
| 15 | HPV66-E | GGAGGAAAAACAATIGCACTGTGAA (SEQ ID NO. 45) | 25 | C |
| 16 | HPV68-E | CGCTATTICACAACCCTGAGG (SEQ ID NO. 46) | 21 | A |

TABLE 1C
Spacer designations affixed to 5′ end of extension primer

| | | Designation (Integrated |
|---|---|---|
| Modifications | Daltons | DNA Technologies |
| Phosphorylation | 80 | /5Phos/ |
| C3 spacer | 138 | /5SpC3/ |
| D spacer | 180 | /5dSp/ |
| Amino Modifier C12 | 264 | /5AmMC12/ |
| Spacer 18 | 344 | /5Sp18/ |
| Amino Modifier C6 dT | 458 | /5AmMC6T |

TABLE 10
Competitor Sequences

| Competitor for: | Sequence |
|---|---|
| HPV16 | ATGTTTCAGGACCCACAGGAGCGACCCAGAAAGTTACCACAGTTATGC |
| | ACAGAGCTGCAAACAATTATACATGATATACTATTAGAATGTGTGTACT |
| | GCAAGCAACAGTTACTGCGACGTG (SEQ ID NO. 47) |
| HPV18 | ATGCATGGACCTAAGGCAACATTGCAAGACTAACATATGTATTGCATTT |
| | AGAGCCCCAAAATGAAATTCCGGTTGACCTTC (SEQ ID NO. 48) |
| HPV31 | AAAGTGGTGAACCGAAAACGCTTAAGCACATAGTATTTTGTGCAAACC |
| | TACAGACGCTTTATGCATCTGCAGAAAGACCTCGGAAATTGCA (SEQ |
| | ID NO. 49) |
| HPV33 | CAAGACACTGAGGAAAAACCACCAACATTGCATGATTTGTGCCAAGCA |
| | TTGGTCACAACATATCAGTTCTAAGAACTACAGTGCGTGGAATG (SEQ |
| | ID NO. 50) |
| HPV35 | ACATGTCAAAAACCGCTGTGTCCAGTTGAAAAGCAAAGACATTTAGAA |
| | GAAAAAAAACGATTCCATAACATCGGTGGATGGTGGACAGGTCGGTGT |
| | ATGTCC (SEQ ID NO. 51) |
| HPV39 | AATCCTGCAGAACGGCCATAGTTTGCAGGTCGCAACACGTGTCCGTTA |
| | AACACCACCTTGCAGGACATTACAATAGCCTGTGTTGACGTTATACGA |
| | CCACTACAGCAAACC (SEQ ID NO. 52) |
| HPV45 | TTGTGGAAAAGTGCATTACAGGATGGCGCGCTTTGACGATCTGACTGA |
| | CTAGCTCTAGTTGCTACCAGATTTGTGCACAGA (SEQ ID NO. 53) |
| HPV51 | AAGGGTTATGACCGAAAACGGTGCATATAAAAGTGCAGTGGTTGACTG |
| | ACTAGCTCTAGTTATGCCTAGGAGCAAGAGGGAAAGACCACGAA (SEQ |
| | ID NO. 54) |
| HPV52 | GAGGATCCAGCAACACGACCCCTCCCGGAGCACGAATTGTGTGAGGT |
| | GCTGGAAGAATTGGTGCATGAAATAAGGCTGCA (SEQ ID NO. 55) |
| HPV56 | TTAACTCCGGAGGAAAAGCAATTGCATTGTGACTGTTTAGCACACATG |
| | CATCTAATGAAAAAAGGTTGGACCGGGTCATGTTT (SEQ ID NO. 56) |
| HPV58 | ACCACGGACATTGCATGATTTGTGTCAGGTACCAAGAGTGTCTGTGCA |
| | TGAAATCGAATTG (SEQ ID NO. 57) |
| HPV59 | ATTGCGAGCCTTACAGCATGTGTGTTTCCTATCACACAGGTATTTGTC |
| | GCCTTTGTGTGCAGCAAACCAGTAACCTGTGGTAACCGATTCGGAAG |
| | GTACAG (SEQ ID NO. 58) |
| HPV66 | CGTTAACACCGGAGGAAAAACAATTGCACTGTGAATTATATAGACGAT |
| | TTCATTATATAGCATATGC (SEQ ID NO. 59) |
| HPV68 | ATGGCGCTATTTCACAACCCTGAGGTGACCTGTGCAGGACATTGACG |
| | GCCATACAAATTGCCAGACACCACATTGCATGACGT (SEQ ID NO. 60) |
| HPV73 | TCCACTGGAAAAGCAAAAGCATGTAGATGAAAAAAAACGGTTTCATCA |
| | AATAGTAGAACAGTGGACCGGACGGTGACGCTGTACCTGGAGACCAT |
| | CTGCAACTG (SEQ ID NO. 61) |
| GAPDH | CAAGAAGGTGGTGAAGCAGACGCCGGAAGGCCCCCTCAAGGGCATC |
| | CTGGGCTACACTGAGCACCAGGTGGTCTCCTCTGACTTCATCAGCGA |
| | CACCCACTCCTCCACCAAAGACGCTCCCCGTGGCATTGGGTCATCGA |
| | CCACTTTGTCAAGCTCA (SEQ ID NO. 62) |

### Validation Using Clinical Samples

Methods and compositions provided herein were used to type a cohort of 226 samples with known outcome. The results showed fourteen samples with an invalid result, as judged by the absence of HPV or GAPDH, and, of the remaining 212, all tests indicated the correct HPV status. See Table 1E. PCR amplification was followed by a primer extension reaction (Sequenom® SensiPLEX™ Kit) and detection of the extension products using mass spectrometry (Sequenom® MassARRAY® System). Exemplary SensiPLEX™ methods are described by Akolekar, R., Farkas, D. H., VanAgtmael, A. L., Bombard, A. T. and Nicolaides, K. H., Fetal sex determination using circulating cell-free fetal DNA (ccffDNA) at 11 to 13 weeks of gestation. Prenatal Diagnosis, (2010) n/a. doi: 10.1002/pd.2582; and Komar (ed.), Single Nucleotide Polymorphisms, Methods in Molecular Biology, Volume: 578 (2008), both of which are hereby incorporated by reference. The temperature for the first PCR reaction was about 60.degree. C. and the temperature for the second primer extension reaction was about 58.degree. C. Synthetic competitor sequences The validity of the results was measured by HC-2 (Qiagen) and Roche Line blot analyses, and shown to give identical calls for the presence and absence of high risk HPV and the genotype. The 14 invalid calls by the SensiPLEX HPV16 method were subsequently called negative by the other methods. Type specific sequencing also was used to confirm the presence of the HPV in the positive samples.

For the results provided in Table 1E, the cohorts were scored using a peak probability of 0.99 for the GAPDH (control) equal to “Adequate” in the case of HPV negative. If any HPV type was present at a 0.99 peak probability, the sample was called positive for that type. A sample that is “positive” for an HPV type does not have to have an “Adequate” GAPDH, as the HPV DNA in a sample can be very much more abundant than the human. Thus the performance of the HPV assays provided herein can be described in terms of each assay's “call rate”. The call rate is a measure of how often an assay predicts the correct outcome. In the case of the HPV, a call rate is the rate at which the presence or absence of HPV is correctly detected in a biological sample. In some embodiments, the call rate for the HPV assays described herein is 80% or greater, 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, or 99% or greater.

TABLE 1E
Summary n = 212 (226-14 invalid)

| | |
|---|---|
| Negative | 197 |
| HPV16 | 6 |
| HPV18 | 2 |
| HPV33 | 1 |
| HPV35 | 4 |
| HPV59 | 1 |
| HPV16 and HPV35 | 1 |

### EXAMPLES OF EMBODIMENTS

Listed hereafter are non-limiting examples of certain embodiments of the technology. In some embodiments, provided is a method, comprising (a) contacting a sample from a subject with five (5) or more amplification primers each specific for a HPV type under amplification conditions, (b) contacting the nucleic acid after step (a) with five (5) or more extension primers specific for amplification products that may be produced by step (a) under extension conditions, (c) detecting the presence, absence or amount of an extension product formed in step (b) whereby the presence, absence and/or amount of the five (5) or more of the HPV types is determined.

In some embodiments, the HPV type is a high-risk or potentially high-risk HPV (e.g., HPV 16, 18, 31, 33, 35, 39, 45, 51, 52, 56, 58, 59, 66, 68, and 73) and in certain embodiments, five (5) or more of the following HPV types are screened: 6, 11, 16, 18, 31, 33, 35, 39, 45, 51, 52, 56, 58, 59, 66, 68, and/or 73. In some embodiments, 5, 6, 7, 8, 9, 10, 11, 12, 13, 14, 15 or more HPV types are assessed (e.g., a matched number of specific primers for amplification (e.g., PCR) and extension are utilized in the assay). In certain embodiments, one or more amplification primers and/or extension primers are mass modified, often in a manner that gives rise to resolution above resolution limits of extension products on a mass spectrogram that includes products specific to five (5) or more HPV types.

Often two primers are utilized for amplification in step (a) (e.g., a forward and reverse primer). In some embodiments, a competitor nucleic acid having a sequence identical to a HPV target nucleic acid subsequence but for one or more nucleotide substitutions, deletions or additions (e.g., 1, 2, 3, 4, 5, 6, 7, 8, 9 or 10 nucleotide substitutions, insertions and/or deletions) is included in the amplification conditions in step (a). In certain embodiments, a competitor nucleic acid is included in step (a) that substantially does not hybridize to a HPV nucleic acid in a sample, but does include one or more stretches of contiguous nucleotides that hybridize to amplification primers (e.g., the competitor nucleic acid includes scrambled versions of HPV sequences). In some embodiments, the competitor nucleic acid is amplified by the amplification primers of step (a), and in certain embodiments, an extension primer abuts the nucleotide substitution of the competitor nucleic acid that presents in the amplification product produced in step (a) from the competitor nucleic acid template. In certain embodiments, the extension conditions include two terminating nucleotides (e.g., dideoxy nucleotides), and sometimes one terminating nucleotide incorporates at the position in an amplification product arising from the competitor nucleic acid corresponding to a nucleotide substitution, and one terminating nucleotide incorporates at the position in an amplification product arising from the HPV nucleic acid corresponding to the position that is substituted in the competitor. In certain embodiments, a competitor nucleic acid is present in step (a) at a concentration of about 50 attomolar to about 2000 attomolar (e.g., about 10, 15, 20, 25, 30, 35, 40, 45, 50, 60, 70, 80, 90, 100, 150, 200, 250, 300, 400, 500, 600, 700, 800, 900, 1000, 1200, 1400, 1600, 1800, 2000 attomolar). In some embodiments, a competitor nucleic acid is included in a different defined amounts in the amplification conditions of step (a) of different assays, whereby the amount of the HPV nucleic acid template in the sample can be quantified (e.g., via a standard curve).

In certain embodiments, the presence or amount of an HPV type is determined when the HPV nucleic acid is present in an assayed sample at a concentration of 50 attomolar (aM) or less (e.g., 40 aM or less, 30 aM or less, 25 aM or less, 20 aM or less, 15 aM or less, 10 aM or less, 5 aM or less, 1 aM or less), or at a copy number of 300 molecules of HPV type nucleic acid or less (e.g., 200 copies or less, 100 copies or less, 50 copies or less, 25 copies or less, 20 copies or less, 15 copies or less, 10 copies or less, 5 copies or less or 1 copy). In some embodiments, presence, absence and/or amount of HPV DNA and/or HPV RNA is detected.

In certain embodiments, a method includes (d) if the presence or amount of a particular HPV type is detected in step (c), screening a sample from the same subject with amplification primers and/or extension primers specific for only the type of HPV detected in step (c), and (e) detecting the presence, absence or amount of the HPV type detected in (c).

In certain embodiments, the presence, absence and/or amount of an HPV type in step (c) or step (e) is determined with a call rate of 80% or greater (e.g., 81, 82, 83, 84, 85, 86, 87, 88, 89, 90, 91, 92, 93, 94, 95, 96, 97, 98, 99%).

In some embodiments, the extension product(s) of step (b) is or are detected by mass spectrometry (e.g., matrix-assisted laser desorption (MALDI) mass spectrometry, electrospray mass spectrometry and others). At times, an extension product is detected by mass spectrometry at a resolution of about 20 Daltons (e.g., about 5 Daltons, 10 Daltons, 15 Daltons, 25 Daltons, 30 Daltons, 35 Daltons, 40 Daltons resolution).

In some embodiments, the sample includes nucleic acid (referred to hereafter as “template nucleic acid”) from a cell or group of cells, and sometimes the cells are from a tissue or fluid from the subject (e.g., described herein). In certain embodiments, the sample includes nucleic acid from a tissue sample or fluid from the subject, and sometimes the cells, tissue sample, or fluid from the subject are different samples or types in steps (a) and (d).

In some embodiments, provided is a composition that includes one or more (a) amplification primers (e.g., Table 1A), (b) extension primers (e.g., Table 1B), (c) competitor nucleic acids (e.g., Table 1D), and/or (d) components for amplification conditions or extension conditions. Also provided in some embodiments are kits that include one or more primers described herein (e.g., amplification primers, extension primers), one or more competitor nucleic acids described herein, components for amplification conditions and/or extension conditions (e.g., extension nucleotides (dNTPs), chain terminating nucleotides (ddNTPs), buffers, polymerase enzymes, etc.), in any suitable combination. In some embodiments, a composition or kit includes 1 to 30 amplification primers (e.g., 2, 4, 6, 8, 10, 12, 14, 16, 18, 20, 22, 24, 26, 28, 30 amplification primers), 1 to 15 extension primers (e.g., 1, 2, 3, 4, 5, 6, 7, 8, 9, 10, 11, 12, 13, 14, 15 extension primers) and/or 1 to 15 competitor nucleic acids (e.g., 1, 2, 3, 4, 5, 6, 7, 8, 9, 10, 11, 12, 13, 14, 15 competitor nucleic acids). In some embodiments a composition or kit includes 1 to 15 sets (e.g., 1, 2, 3, 4, 5, 6, 7, 8, 9, 10, 11, 12, 13, 14, 15 sets) of nucleic acid reagents, where each set includes two amplification primers, one extension primer and optionally one competitor nucleic acid, and where each set is specific for a particular HPV type.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claims that follow.

1-41. (canceled)

42. A method for detecting the presence or absence of HPV DNA in a biological sample, comprising:
a) conducting an amplification reaction of at least a portion of the DNA from the biological sample;
b) conducting a primer extension reaction in the presence of at least one extension primer for said known HPV type and at least one terminating nucleotide, wherein at least one extension primer sequence is selected from the group consisting of
(SEQ ID NO. 31)
AGGCAACAITGCAAGAC;
(SEQ ID NO. 32)
CAGGATGGCGCGCITTGACGATC;
(SEQ ID NO. 33)
CAGGACATTACAAIAGCCTGTGT;
(SEQ ID NO. 34)
TGCAGCAAACCAGIAACCTG;
(SEQ ID NO. 35)
/5AmMC6/GAAAAGCAAITGCATTGTGAC;
(SEQ ID NO. 36)
GGTCTCCTCTGACTTCA;
(SEQ ID NO. 37)
GTGCATAIAAAAGTGCAGTGGT;
(SEQ ID NO. 38)
TGCAAACCIACAGACGCC;
(SEQ ID NO. 39)
CATCGGIGGACGGTGG;
(SEQ ID NO. 40)
ATGATTIGTGCCAAGCATTGG;
(SEQ ID NO. 41)
/5AmMC6/ACATTGCATGAITTGTGTC;
(SEQ ID NO. 42)
GTGTGAGGIGCTGGAAGAAT;
(SEQ ID NO. 43)
GAAAAAAAACGGITTCATCAAATAGC;
(SEQ ID NO. 44)
TGCACAGAGCIGCAAACAA;
(SEQ ID NO. 45)
GGAGGAAAAACAATIGCACTGTGAA;
(SEQ ID NO. 46)
CGCTATTICACAACCCTGAGG,
and further wherein “I” represents a deoxyinosine or other nucleotide substitutes, and /5AmMC6/ is a mass modifier; and
c) detecting HPV DNA in a biological sample, wherein the presence or absence of known HPV extension products from step b) is correlated with detecting the presence or absence of HPV DNA.

43. The method of claim 42, wherein detecting HPV DNA comprises detecting the presence or absence of five or more HPV types.

44. The method of claim 43, wherein one or more of the five or more HPV types are a high-risk or potentially high-risk HPV type.

45. The method of claim 44, wherein one or more of the five or more HPV types are chosen from HPV type 6, 11, 16, 18, 31, 33, 35, 39, 45, 51, 52, 56, 58, 59, 66, 68 and 73.

46. The method of claim 43, wherein the presence, absence of 6, 7, 8, 9, 10, 11, 12, 13, 14, 15 or more HPV types are assessed.

47. The method of claim 42, wherein the first amplification comprises at least one matched set of forward and reverse primer sequences for said known HPV type consisting of the primers listed below, wherein each primer of the primer pair (i) comprises one of the full length nucleotide sequences hereafter, (ii) comprises one of the non-underlined nucleotide sequences hereafter, or (iii) comprises one of the non-underlined nucleotide sequences hereafter and a tag nucleotide sequence:
(SEQ ID NO 1)
ACGTTGGATGATGTTICAGGACCCACAGGA, HPV16
(SEQ ID NO 16)
ACGTTGGATGCACGTCGCAGIAACTGTTGC;
(SEQ ID NO 2)
ACGTTGGATGATGCATGGACCIAAGGCAAC, HPV18
(SEQ ID NO 17)
ACGTTGGATGGAAGGICAACCGGAATTTCA;
(SEQ ID NO 3)
ACGTTGGATGAAAGTGGTGAICCGAAAACG, HPV31
(SEQ ID NO 18)
ACGTTGGATGTTTCCGAGGICTTTCTGCAG;
(SEQ ID NO 4)
ACGTTGGATGCAAGACACIGAGGAAAAACCAC, HPV33
(SEQ ID NO 19)
ACGTTGGATGCATTCCACGCACIGTAGTTC;
(SEQ ID NO 5)
ACGTTGGATGACATGTCAAIAACCGCTGTG, HPV35
(SEQ ID NO 20)
ACGTTGGATGAACAGGACAIACACCGACCT;
(SEQ ID NO 6)
ACGTTGGATGAATCCIGCAGAACGGCCATA, HPV39
(SEQ ID NO 21)
ACGTTGGATGGGTTTGCTGIAGTGGTCGT;
(SEQ ID NO 7)
ACGTTGGATGTTGTGGAAAAGIGCATTACAGG, HPV45
(SEQ ID NO 22)
ACGTTGGATGTCTGTGCACAAAICTGGTAGC;
(SEQ ID NO 8)
ACGTTGGATGAAGGGTTAIGACCGAAAACG, HPV51
(SEQ ID NO 23)
ACGTTGGATGTTCGTGGTCITTCCCTCTTG;
(SEQ ID NO 9)
ACGTTGGATGGAGGATCCIGCAACACGAC, HPV52
(SEQ ID NO 24)
ACGTTGGATGTGCAGCCTIATTTCATGCAC;
(SEQ ID NO 10)
ACGTTGGATGTTAACTCCGGIGGAAAAGC, HPV56
(SEQ ID NO 25)
ACGTTGGATGAAACAIGACCCGGTCCAAC;
(SEQ ID NO 11)
ACGTTGGATGACCACGGACAITGCATGATT, HPV58
(SEQ ID NO 26)
ACGTTGGATGCAATTCGATTICATGCACAGA;
(SEQ ID NO 12)
ACGTTGGATGATTGCGAGCCTIACAGCA, HPV59
(SEQ ID NO 27)
ACGTTGGATGCTGTACCTICCGAATCGG;
(SEQ ID NO 13)
ACGTTGGATGCGTIAACACCGGAGGAAAAA, HPV66
(SEQ ID NO 28)
ACGTTGGATGTGCATATGCTAIATAATGAAATCGTC;
(SEQ ID NO 14)
ACGTTGGATGAATGGCGCIATTTCACAACC, HPV68
(SEQ ID NO 29)
ACGTTGGATGACGTCAIGCAATGTGGTGTC
(SEQ ID NO 15)
ACGTTGGATGTCCACTGGAIAAGCAAAAGC,); HPV73
(SEQ ID NO 30)
ACGTTGGATGCAGTTGCAGAIGGTCTCCAG,
wherein “I’ represents a deoxyinosine.

48. The method of claim 42, wherein the amplification conditions in step (a) comprise a competitor nucleic acid having a sequence identical to a nucleotide subsequence of the known HPV types but for one or more nucleotide substitutions, deletions or additions and the method comprises quantification of the amount of a HPV nucleic acid in a sample.

49. The method of claim 48, wherein the competitor nucleic acid substantially does not hybridize to a HPV nucleic acid in a sample, and includes one or more stretches of contiguous nucleotides that hybridize to amplification primers.

50. The method of claim 48, wherein the competitor nucleic acid is amplified by the amplification primers in step (a).

51. The method of claim 48, wherein an extension primer abuts a nucleotide substitution of the competitor nucleic acid that is present in the amplification product produced in step (a) from the competitor nucleic acid template.

52. The method of claim 51, wherein the extension conditions include two terminating nucleotides.

53. The method of claim 52, wherein the terminating nucleotides are different dideoxy nucleotides.

54. The method of claim 53, wherein one dideoxynucleotide incorporates at a position in an amplification product corresponding to a nucleotide substitution in the competitor nucleic acid.

55. The method of claim 48, wherein at least one competitor sequence is selected from the group consisting of:
(SEQ ID NO. 47)
ATGTTTCAGGACCCACAGGAGCGACCCAGAAAGTTACCACAGTTATGCAC
AGAGCTGCAAACAATTATACATGATATACTATTAGAATGTGTGTACTGCA
AGCAACAGTTACTGCGACGTG;
(SEQ ID NO. 48)
ATGCATGGACCTAAGGCAACATTGCAAGACTAACATATGTATTGCATTTA
GAGCCCCAAAATGAAATTCCGGTTGACCTTC;
(SEQ ID NO. 49)
AAAGTGGTGAACCGAAAACGCTTAAGCACATAGTATTTTGTGCAAACCTA
CAGACGCTTTATGCATCTGCAGAAAGACCTCGGAAATTGCA;
(SEQ ID NO. 50)
CAAGACACTGAGGAAAAACCACCAACATTGCATGATTTGTGCCAAGCATT
GGTCACAACATATCAGTTCTAAGAACTACAGTGCGTGGAATG;
(SEQ ID NO. 51)
ACATGTCAAAAACCGCTGTGTCCAGTTGAAAAGCAAAGACATTTAGAAGA
AAAAAAACGATTCCATAACATCGGTGGATGGTGGACAGGTCGGTGTATGT
CC;
(SEQ ID NO. 52)
AATCCTGCAGAACGGCCATAGTTTGCAGGTCGCAACACGTGTCCGTTAAA
CACCACCTTGCAGGACATTACAATAGCCTGTGTTGACGTTATACGACCAC
TACAGCAAACC;
(SEQ ID NO. 53)
TTGTGGAAAAGTGCATTACAGGATGGCGCGCTTTGACGATCTGACTGACT
AGCTCTAGTTGCTACCAGATTTGTGCACAGA;
(SEQ ID NO. 54)
AAGGGTTATGACCGAAAACGGTGCATATAAAAGTGCAGTGGTTGACTGAC
TAGCTCTAGTTATGCCTAGGAGCAAGAGGGAAAGACCACGAA;
(SEQ ID NO. 55)
GAGGATCCAGCAACACGACCCCTCCCGGAGCACGAATTGTGTGAGGTGCT
GGAAGAATTGGTGCATGAAATAAGGCTGCA;
(SEQ ID NO. 56)
TTAACTCCGGAGGAAAAGCAATTGCATTGTGACTGTTTAGCACACATGCA
TCTAATGAAAAAAGGTTGGACCGGGTCATGTTT;
(SEQ ID NO. 57)
ACCACGGACATTGCATGATTTGTGTCAGGTACCAAGAGTGTCTGTGCATG
AAATCGAATTG;
(SEQ ID NO. 58)
ATTGCGAGCCTTACAGCATGTGTGTTTCCTATCACACAGGTATTTGTCGC
CTTTGTGTGCAGCAAACCAGTAACCTGTGGTAACCGATTCGGAAGGTACA
G;
(SEQ ID NO. 59)
CGTTAACACCGGAGGAAAAACAATTGCACTGTGAATTATATAGACGATTT
CATTATATAGCATATGC;
(SEQ ID NO. 60)
ATGGCGCTATTTCACAACCCTGAGGTGACCTGTGCAGGACATTGACGGCC
ATACAAATTGCCAGACACCACATTGCATGACGT;
(SEQ ID NO. 61)
TCCACTGGAAAAGCAAAAGCATGTAGATGAAAAAAAACGGTTTCATCAAA
TAGTAGAACAGTGGACCGGACGGTGACGCTGTACCTGGAGACCATCTGCA
ACTG;
and
(SEQ ID NO. 62)
CAAGAAGGTGGTGAAGCAGACGCCGGAAGGCCCCCTCAAGGGCATCCTGG
GCTACACTGAGCACCAGGTGGTCTCCTCTGACTTCATCAGCGACACCCAC
TCCTCCACCAAAGACGCTCCCCGTGGCATTGGGTCATCGACCACTTTGTC
AAGCTCA.

56. The method of claim 48, wherein the presence or amount of an HPV type is determined when the HPV nucleic acid is present in an assayed sample at a concentration of about 50 attomolar or less.

57. The method of claim 42, wherein the extension product of step (b) is detected by mass spectrometry.

58. A method for detecting the presence, absence or amount of five (5) or more Human Papilloma Virus (HPV) types, comprising:
(a) contacting a sample from a subject with five (5) or more amplification primers each specific for a HPV type under amplification conditions, thereby generating an amplification reaction,
(b) contacting the amplification reaction after step (a) with five (5) or more extension primers specific for amplification products that may be produced in step (a) under extension conditions,
(c) detecting the presence, absence or amount of an extension product formed in step (b) whereby the presence, absence and/or amount of the five (5) or more of the HPV types is determined.

59. The method of claim 58, wherein one or more of the five or more HPV types are chosen from HPV type 6, 11, 16, 18, 31, 33, 35, 39, 45, 51, 52, 56, 58, 59, 66, 68 and 73.

60. The method of claim 58, wherein the presence, absence of 6, 7, 8, 9, 10, 11, 12, 13, 14, 15 or more HPV types are assessed.

61. A kit comprising (a) one or more amplification primers chosen from Table 1A, (b) one or more extension primers chosen from Table 1B, (c) optionally one or more competitor nucleic acids chosen from Table 1D, and/or (d) one or more components for amplification and/or extension.
